(19) United States
(12) Patent Application Publication
(10) Pub. No.: US 2009/0105250 A1
(43) Pub. Date: Apr. 23, 2009

(54) COMPOUNDS AND COMPOSITIONS AS PROTEIN KINASE INHIBITORS

(75) Inventors: Taebo Sim, San Diego, CA (US); Nathanael Schiander Gray, San Diego, CA (US); Hyun Soo Lee, San Diego, CA (US); Yi Liu, San Diego, CA (US); Pingda Ren, San Diego, CA (US); Shuli You, San Diego, CA (US); Qiong Zhang, San Diego, CA (US); Qiang Ding, San Diego, CA (US); Xia Wang, San Diego, CA (US); Songchun Jiang, San Diego, CA (US); Pamela A. Albaugh, Carlsbad, CA (US)

Correspondence Address: GENOMICS INSTITUTE OF THE;NOVARTIS RESEARCH FOUNDATION, 10675 JOHN JAY HOPKINS DRIVE, SUITE E225, SAN DIEGO, CA, 92121-1127, US

(73) Assignee: IRM LLC, Hamilton, BM (BM)

(21) Appl. No.: 11/814,912

(22) PCT Filed: Jan. 19, 2006
(86) PCT No.: PCT/US06/02266

§ 371 Date: Apr. 3, 2008

Related U.S. Application Data
(60) Provisional application No. 60/647,606, filed on Jan. 26, 2005.

(51) Int. Cl. A61K 31/427; C07D 277/56; C07D 417/14; C07D 417/12; A61K 31/454; A61K 31/506; A61K 31/5377; A61K 31/426

(52) U.S. Cl. 514/235.8; 548/195; 544/295; 544/369; 544/133; 544/328; 544/122; 546/209; 514/252.19; 514/236.8; 514/370; 514/256; 514/326; 514/254.02

(57) ABSTRACT

The invention provides a novel class of compounds, pharmaceutical compositions comprising such compounds and methods of using such compounds to treat or prevent diseases or disorders associated with abnormal or deregulated kinase activity, particularly diseases or disorders that involve abnormal activation of the Abl, Bcr-Abl, FGFR3, PDGFRβ and b-Raf kinases.

### CROSS-REFERENCE TO RELATED APPLICATIONS

This application claims the benefit of priority to U.S. Provisional Patent Application No. 60/647,606, filed 25 Jan. 2005. The full disclosure of this application is incorporated herein by reference in its entirety and for all purposes.

### BACKGROUND OF THE INVENTION

1. Field of the Invention

The invention provides a novel class of compounds, pharmaceutical compositions comprising such compounds and methods of using such compounds to treat or prevent diseases or disorders associated with abnormal or deregulated kinase activity, particularly diseases or disorders that involve abnormal activation of the Abl, Bcr-Abl, FGFR3, PDGFRβ, Flt3 and b-Raf kinases.

2. Background

The protein kinases represent a large family of proteins, which play a central role in the regulation of a wide variety of cellular processes and maintaining control over cellular function. A partial, non-limiting, list of these kinases include: receptor tyrosine kinases such as platelet-derived growth factor receptor kinase (PDGF-R) and the fibroblast growth factor receptor, FGFR3; non-receptor tyrosine kinases such Abl and the fusion kinase BCR-Abl; and serine/threonine kinases such as b-RAF, SGK, MAP kinases (e.g., MKK4, MKK6, etc.) and SAPK2α, and SAPK2β. Aberrant kinase activity has been observed in many disease states including benign and malignant proliferative disorders as well as diseases resulting from inappropriate activation of the immune and nervous systems.

The novel compounds of this invention inhibit the activity of one or more protein kinases and are, therefore, expected to be useful in the treatment of kinase-associated diseases.

### SUMMARY OF THE INVENTION

In one aspect, the present invention provides compounds of Formula I:

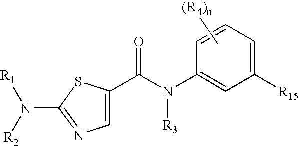

in which:

n is selected from 0, 1, 2, 3 and 4;

R₁ is selected from hydrogen, C₁₋₆alkyl, C₆₋₁₀aryl-C₀₋₄alkyl, C₅₋₁₀heteroaryl-C₀₋₄alkyl, C₃₋₁₂cycloalkyl-C₀₋₄alkyl, C₃₋₈heterocycloalkyl-C₀₋₄alkyl and —XNR₇R₈;

wherein any aryl, heteroaryl, cycloalkyl or heterocycloalkyl of R₁ is optionally substituted with 1-3 radicals independently selected from halo, C₁₋₆alkyl, halo-substituted-C₁₋₆alkyl, C₁₋₆alkoxy, halo-substituted-C₁₋₆alkoxy, C₁₋₆alkylthio, halo-substituted-C₁₋₆alkylthio, —XNR₇R₈, —XNR₇XNR₇R₈, —XNR₇R₉, C₆₋₁₀aryl-C₀₋₄alkyl, C₅₋₁₀heteroaryl-C₀₋₄alkyl, C₃₋₁₂cycloalkyl-C₀₋₄-alkyl and C₃₋₈heterocycloalkyl-C₀₋₄alkyl; wherein any aryl, heteroaryl, cycloalkyl or heterocycloalkyl substituents on R₁ can be optionally substituted by 1 to 3 radicals independently selected from halo, C₁₋₆alkyl, halo-substituted-C₁₋₆alkyl, hydroxy-substituted-C₁₋₆alkyl, C₁₋₆alkoxy and halo-substituted-C₁₋₆alkoxy; and wherein any alkyl of R₁ can have a methylene replaced with O;

wherein each X is independently selected from a bond and C₁₋₆alkylene; R₇ and R₈ are independently selected from hydrogen and C₁₋₆alkyl; wherein any methylene of R₇ and R₈ can be replaced with O; wherein R₉ is selected from C₆₋₁₀aryl-C₀₋₄-alkyl, C₅₋₁₀heteroaryl-C₀₋₄alkyl, C₃₋₁₂cycloalkyl-C₀₋₄alkyl and C₃₋₈heterocycloalkyl-C₀₋₄alkyl;

R₂ is selected from hydrogen and C₁₋₆alkyl;

R₃ is selected from hydrogen and C₁₋₆alkyl;

R₄ is selected from halo, C₁₋₆alkyl, halo-substituted-C₁₋₆alkyl, C₁₋₆alkoxy, halo-substituted-C₁₋₆alkoxy, C₁₋₆alkylthio and halo-substituted-C₁₋₆alkylthio;

R₁₅ is selected from —NR₅Y(O)R₆ and —Y(O)NR₅R₆; wherein

Y is selected from C, S, S(O), P and P(O);

R₅ is selected from hydrogen and C₁₋₆alkyl; and

R₆ is selected from C₆₋₁₀aryl, C₅₋₁₀heteroaryl, C₃₋₁₂cycloalkyl and C₃₋₈heterocycloalkyl; wherein said aryl, heteroaryl, cycloalkyl or heterocycloalkyl of R₆ is optionally substituted with 1 to 3 substituents independently selected from halo, C₁₋₆alkyl, halo-substituted-C₁₋₆alkyl, C₁₋₆alkoxy, halo-substituted-C₁₋₆alkoxy, C₁₋₆alkylthio, halo-substituted-C₁₋₆alkylthio, C₆₋₁₀aryl-C₀₋₄alkyl, C₅₋₁₀heteroaryl-C₀₋₄alkyl, C₃₋₁₂cycloalkyl-C₀₋₄alkyl, C₃₋₈heterocycloalkyl-C₀₋₄alkoxy and C₃₋₈heterocycloalkyl-C₀₋₄alkyl; wherein the aryl, heteroaryl, cycloalkyl or heterocycloalkyl substituents on R₆ can be optionally be further substituted by 1 to 3 radicals independently selected from hydroxy, halo, C₁₋₆alkyl, halo-substituted-C₁₋₆alkyl, hydroxy-substituted-C₁₋₆alkyl, C₁₋₆alkoxy and halo-substituted-C₁₋₆alkoxy; and the N-oxide derivatives, prodrug derivatives, protected derivatives, individual isomers and mixture of isomers thereof; and the pharmaceutically acceptable salts and solvates (e.g. hydrates) of such compounds.

In a second aspect, the present invention provides a pharmaceutical composition which contains a compound of Formula I or a N-oxide derivative, individual isomers and mixture of isomers thereof; or a pharmaceutically acceptable salt thereof, in admixture with one or more suitable excipients.

In a third aspect, the present invention provides a method of treating a disease in an animal in which inhibition of kinase activity, particularly Abl, Bcr-Abl, FGFR3, PDGFRβ, Flt3 and b-Raf activity, can prevent, inhibit or ameliorate the pathology and/or symptomology of the diseases, which method comprises administering to the animal a therapeutically effective amount of a compound of Formula I or a N-oxide derivative, individual isomers and mixture of isomers thereof, or a pharmaceutically acceptable salt thereof.

In a fourth aspect, the present invention provides the use of a compound of Formula I in the manufacture of a medicament for treating a disease in an animal in which kinase activity, particularly Abl, Bcr-Abl, FGFR3, PDGFRβ, Flt3 and b-Raf activity, contributes to the pathology and/or symptomology of the disease.

In a fifth aspect, the present invention provides a process for preparing compounds of Formula I and the N-oxide derivatives, prodrug derivatives, protected derivatives, individual isomers and mixture of isomers thereof, and the pharmaceutically acceptable salts thereof.

### DETAILED DESCRIPTION OF THE INVENTION

### Definitions

“Alkyl” as a group and as a structural element of other groups, for example halo-substituted-alkyl and alkoxy, can be either straight-chained or branched. C₁₋₄-alkoxy includes, methoxy, ethoxy, and the like. Halo-substituted alkyl includes trifluoromethyl, pentafluoroethyl, and the like.

“Aryl” means a monocyclic or fused bicyclic aromatic ring assembly containing six to ten ring carbon atoms. For example, aryl may be phenyl or naphthyl, preferably phenyl. “Arylene” means a divalent radical derived from an aryl group.

“Heteroaryl” is as defined for aryl above where one or more of the ring members is a heteroatom. For example heteroaryl includes pyridyl, indolyl, indazolyl, quinoxalinyl, quinolinyl, benzofuranyl, benzopyranyl, benzothiopyranyl, benzo[1,3]dioxole, imidazolyl, benzo-imidazolyl, pyrimidinyl, furanyl, oxazolyl, isoxazolyl, triazolyl, tetrazolyl, pyrazolyl, thienyl, etc.

“Cycloalkyl” means a saturated or partially unsaturated, monocyclic, fused bicyclic or bridged polycyclic ring assembly containing the number of ring atoms indicated. For example, C₃₋₁₀cycloalkyl includes cyclopropyl, cyclobutyl, cyclopentyl, cyclohexyl, etc.

“Heterocycloalkyl” means cycloalkyl, as defined in this application, provided that one or more of the ring carbons indicated, are replaced by a moiety selected from —O—, —N═, —NR—, —C(O)—, —S—, —S(O)— or —S(O)₂—, wherein R is hydrogen, C₁₋₄alkyl or a nitrogen protecting group. For example, C₃₋₈heterocycloalkyl as used in this application to describe compounds of the invention includes morpholino, pyrrolidinyl, pyrrolidinyl-2-one, piperazinyl, piperidinyl, piperidinylone, 1,4-dioxa-8-aza-spiro[4.5]dec-8-yl, etc.

“Halogen” (or halo) preferably represents chloro or fluoro, but may also be bromo or iodo.

“Kinase Panel” is a list of kinases comprising Abl(human), Abl(T315I), JAK2, JAK3, ALK, JNK1α1, ALK4, KDR, Aurora-A, Lck, Blk, MAPK1, Bmx, MAPKAP-K2, BRK, MEK1, CaMKII(rat), Met, CDK1/cyclinB, p70S6K, CHK2, PAK2, CK1, PDGFRα, CK2, PDK1, c-kit, Pim-2, c-RAF, PKA(h), CSK, PKBα, cSrc, PKCα, DYRK2, Plk3, EGFR, ROCK-I, Fes, Ron, FGFR3, Ros, Flt3, SAPK2α, Fms, SGK, Fyn, SIK, GSK3β, Syk, IGF-1R, Tie-2, IKKβ, TrKB, IR, WNK3, IRAK4, ZAP-70, ITK, AMPK(rat), LIMK1, Rsk2, Axl, LKB1, SAPK2β, BrSK2, Lyn (h), SAPK3, BTK, MAPKAP-K3, SAPK4, CaMKIV, MARK1, Snk, CDK2/cyclinA, MINK, SRPK1, CDK3/cyclinE, MKK4(m), TAK1, CDK5/p25, MKK6(h), TBK1, CDK6/cyclinD3, MLCK, TrkA, CDK7/cyclinH/MAT1, MRCKβ, TSSK1, CHK1, MSK1, Yes, CK1d, MST2, ZIPK, c-Kit (D816V), MuSK, DAPK2, NEK2, DDR2, NEK6, DMPK, PAK-4, DRAK1, PAR-1Bα, EphA1, PDGFRβ, EphA2, Pim-1, EphA5, PKBβ, EphB2, PKCβI, EphB4, PKCδ, FGFR1, PKCη, FGFR2, PKCθ, FGFR4, PKD2, Fgr, PKG1β, Flt1, PRK2, Hck, PYK2, HIPK2, Ret, IKKα, RIPK2, IRR, ROCK-II(human), JNK2α2, Rse, JNK3, Rsk1(h), PI3 Kγ, PI3 Kδ and PI3-Kβ. Compounds of the invention are screened against the kinase panel (wild type and/or mutation thereof) and inhibit the activity of at least one of said panel members.

“Mutant forms of BCR-Abl” means single or multiple amino acid changes from the wild-type sequence. Mutations in BCR-ABL act by disrupting critical contact points between protein and inhibitor (for example, Gleevec, and the like), more often, by inducing a transition from the inactive to the active state, i.e. to a conformation to which BCR-ABL and Gleevec is unable to bind. From analyses of clinical samples, the repertoire of mutations found in association with the resistant phenotype has been increasing slowly but inexorably over time. Mutations seem to cluster in four main regions. One group of mutations (G250E, Q252R, Y253F/H, E255K/V) includes amino acids that form the phosphate-binding loop for ATP (also known as the P-loop). A second group (V289A, F311L, T315I, F317L) can be found in the Gleevec binding site and interacts directly with the inhibitor via hydrogen bonds or Van der Waals' interactions. The third group of mutations (M351T, E355G) clusters in close proximity to the catalytic domain. The fourth group of mutations (H396R/P) is located in the activation loop, whose conformation is the molecular switch controlling kinase activation/inactivation. BCR-ABL point mutations associated with Gleevec resistance detected in CML and ALL patients include: M224V, L248V, G250E, G250R, Q252R, Q252H, Y253H, Y253F, E255K, E255V, D276G, T277A, V289A, F311L, T315I, T315N, F317L, M343T, M315T, E355G, F359V, F359A, V379I, F382L, L387M, L387F, H396P, H396R, A397P, S417Y, E459K, and F486S (Amino acid positions, indicated by the single letter code, are those for the GenBank sequence, accession number AAB60394, and correspond to ABL type 1a; Martinelli et al., Haematologica/The Hematology Journal, 2005, April; 90-4). Unless otherwise stated for this invention, Bcr-Abl refers to wild-type and mutant forms of the enzyme.

“Treat”, “treating” and “treatment” refer to a method of alleviating or abating a disease and/or its attendant symptoms.

### DESCRIPTION OF THE PREFERRED EMBODIMENTS

The present invention provides compounds, compositions and methods for the treatment of kinase related disease, particularly Abl, Bcr-Abl, FGFR3, PDGFRβ, Flt3 and b-Raf kinase related diseases. For example, leukemia and other proliferation disorders related to BCR-Abl can be treated through the inhibition of wild type and mutant forms of Bcr-Abl.

In one embodiment, with reference to compounds of Formula I, are compounds of Formula Ia:

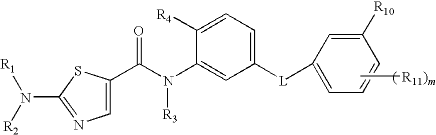

in which:

m is selected from 0 and 1;

R₁ is selected from hydrogen, C₁₋₆alkyl, C₆₋₁₀aryl-C₀₋₄alkyl, C₅₋₁₀heteroaryl-C₀₋₄alkyl, C₃₋₁₂cycloalkyl-C₀₋₄alkyl, C₃₋₈heterocycloalkyl-C₀₋₄alkyl and —XNR₇R₈;

wherein any aryl, heteroaryl, cycloalkyl or heterocycloalkyl of R₁ is optionally substituted with 1 to 3 radicals independently selected from C₁₋₆alkyl, —XNR₇R₈, —XNR₇XNR₇R₈, —XNR₇R₉, C₅₋₁₀heteroaryl-C₀₋₄alkyl and C₃₋₈heterocycloalkyl-C₀₋₄alkyl; wherein any heteroaryl or heterocycloalkyl substituents on R₁ can be optionally substituted by 1 to 3 radicals independently selected from C₁₋₆alkyl and hydroxy-substituted-C₁₋₆alkyl; and wherein any alkyl of R₁ can have a methylene replaced with O;

wherein each X is independently selected from a bond and C₁₋₆alkylene; R₇ and R₉ are independently selected from hydrogen and C₁₋₆alkyl; wherein any methylene of R₇ and R₈ can be replaced with O; wherein R₉ is C₃₋₁₂cycloalkyl-C₀₋₄alkyl;

R₂ is selected from hydrogen and C₁₋₆alkyl;

R₃ is selected from hydrogen and C₁₋₆alkyl;

R₄ is selected from halo, C₁₋₆alkyl, halo-substituted-C₁₋₆alkyl, C₁₋₆alkoxy and halo-substituted-C₁₋₆alkoxy;

L is selected from —NR₅C(O)— and —C(O)NR₅—;

R₅ is selected from hydrogen and C₁₋₆alkyl; and

R₁₀ is halo-substituted-C₁₋₆alkyl; and

R₁₁ is selected from hydrogen, halo, C₅₋₁₀heteroaryl and C₃₋₈heterocycloalkyl; wherein the heteroaryl or heterocycloalkyl substituents on R₁₀ can be optionally substituted by 1 to 3 radicals independently selected from hydroxy and C₁₋₆alkyl.

In another embodiment, R₁ is selected from hydrogen, methyl, isopropyl, imidazolyl-propyl, piperazinyl-propyl, pyridinyl, diethyl-amino-propyl, hydroxy-ethyl, pyrimidinyl, morpholino-propyl, phenyl, cyclopropyl, morpholino-ethyl, benzyl and morpholino; wherein any pyridinyl, imidazolyl, piperazinyl or pyrimidinyl of R₁ is optionally substituted with 1 to 3 radicals independently selected from methyl, methyl-amino, dimethyl-amino-methyl, cyclopropyl-amino, hydroxy-ethyl-amino, diethyl-amino-propyl-amino, pyrrolidinyl-methyl, morpholino, morpholino-methyl, piperazinyl methyl and piperazinyl; wherein any morpholino and piperazinyl substituent of R₁ is optionally further substituted by a radical selected from methyl, hydroxy-ethyl and ethyl; R₂, R₃ and R₅ are each hydrogen; and R₄ is methyl.

In another embodiment, m is selected from 0 and 1; R₁₀ is trifluoromethyl; and R₁ is selected from: halo; morpholino-methyl; piperazinyl optionally substituted with methyl, ethyl or hydroxyethyl; piperazinyl-methyl optionally substituted with methyl or ethyl; imidazolyl optionally substituted with methyl; pyrrolidinyl-methoxy; and piperidinyl optionally substituted with hydroxy.

Preferred compounds of the invention are selected from: 2-(3-Diethylaminopropylamino)-thiazole-5-carboxylic acid [2-methyl-5-(3-trifluoromethyl-benzoylamino)-phenyl]-amide; 2-{6-[4-(2-Hydroxyethyl)-piperazin-1-yl]-2-methylpyrimidin-4-ylamino}-thiazole-5-carboxylic acid [2-methyl-5-(3-trifluoromethylbenzoylamino)-phenyl]-amide; 2-(2-Hydroxy-ethylamino)-thiazole-5-carboxylic acid {5-[3-(4-ethyl-piperazin-1-yl)-5-trifluoromethyl-benzoylamino]-2-methyl-phenyl}-amide; 2-{6-[4-(2-Hydroxy-ethyl)-piperazin-1-yl]-2-methyl-pyrimidin-4-ylamino}-thiazole-5-carboxylic acid [2-methyl-5-(3-trifluoromethyl-benzoylamino)-phenyl]-amide; 2-(3-Morpholin-4-yl-propylamino)-thiazole-5-carboxylic acid [2-methyl-5-(3-trifluoromethyl-benzoylamino)-phenyl]-amide; 2-(3-Diethylamino-propylamino)-thiazole-5-carboxylic acid [2-methyl-5-(3-trifluoromethyl-benzoylamino)-phenyl]-amide; 2-Phenylamino-thiazole-5-carboxylic acid [2-methyl-5-(3-trifluoromethyl-benzoylamino)-phenyl]-amide; 2-(2-Hydroxy-ethylamino)-thiazole-5-carboxylic acid {2-methyl-5-[3-(4-methyl-imidazol-1-yl)-5-trifluoromethyl-benzoylamino]-phenyl}-amide; 2-(3-Diethylamino-propylamino)-thiazole-5-carboxylic acid {2-methyl-5-[3-(4-methyl-imidazol-1-yl)-5-trifluoromethyl-benzoylamino]-phenyl}-amide; 2-(3-Morpholin-4-yl-propylamino)-thiazole-5-carboxylic acid {2-methyl-5-[3-(4-methyl-imidazol-1-yl)-5-trifluoromethyl-benzoylamino]-phenyl}-amide; 2-[6-(4-Ethyl-piperazin-1-yl)-2-methyl-pyrimidin-4-ylamino]-thiazole-5-carboxylic acid [2-methyl-5-(3-trifluoromethyl-benzoylamino)-phenyl]-amide; 2-(6-Cyclopropylamino-2-methyl-pyrimidin-4-ylamino)-thiazole-5-carboxylic acid [2-methyl-5-(3-trifluoromethyl-benzoylamino)-phenyl]-amide; 2-[6-(2-Hydroxy-ethylamino)-2-methyl-pyrimidin-4-ylamino]-thiazole-5-carboxylic acid [2-methyl-5-(3-trifluoromethyl-benzoylamino)-phenyl]-amide; 2-[6-(3-Diethylamino-propylamino)-2-methyl-pyrimidin-4-ylamino]-thiazole-5-carboxylic acid [2-methyl-5-(3-trifluoromethyl-benzoylamino)-phenyl]-amide; 2-(2-Methyl-6-morpholin-4-yl-pyrimidin-4-ylamino)-thiazole-5-carboxylic acid [2-methyl-5-(3-trifluoromethyl-benzoylamino)-phenyl]-amide; 2-(2-Hydroxy-ethylamino)-thiazole-5-carboxylic acid {5-[3-(4-hydroxy-piperidin-1-yl)-5-trifluoromethyl-benzoylamino]-2-methyl-phenyl}-amide; 2-Cyclopropylamino-thiazole-5-carboxylic acid {5-[3-(4-hydroxy-piperidin-1-yl)-5-trifluoromethyl-benzoylamino]-2-methyl-phenyl}-amide; 2-(2-Morpholin-4-yl-ethylamino)-thiazole-5-carboxylic acid {5-[3-(4-hydroxy-piperidin-1-yl)-5-trifluoromethyl-benzoylamino]-2-methyl-phenyl}-amide; 2-Cyclopropylamino-thiazole-5-carboxylic acid {5-[3-(4-ethyl-piperazin-1-yl)-5-trifluoromethyl-benzoylamino]-2-methyl-phenyl}-amide; 2-(2-Hydroxy-ethylamino)-thiazole-5-carboxylic acid {5-[3-(4-ethyl-piperazin-1-yl)-5-trifluoromethyl-benzoylamino]-2-methyl-phenyl}-amide; 2-Benzylamino-thiazole-5-carboxylic acid {5-[3-(4-ethyl-piperazin-1-yl)-5-trifluoromethyl-benzoylamino]-2-methyl-phenyl}-amide, 2-(2-Morpholin-4-yl-ethylamino)-thiazole-5-carboxylic acid {5-[3-(4-ethyl-piperazin-1-yl)-5-trifluoromethyl-benzoylamino]-2-methyl-phenyl}-amide; 2-[6-(2-Hydroxy-ethylamino)-2-methyl-pyrimidin-4-ylamino]-thiazole-5-carboxylic acid {5-[3-(4-ethyl-piperazin-1-yl)-5-trifluoromethyl-benzoylamino]-2-methyl-phenyl}-amide; 2-(6-Cyclopropylamino-2-methyl-pyrimidin-4-ylamino)-thiazole-5-carboxylic acid {5-[3-(4-ethyl-piperazin-1-yl)-5-trifluoromethyl-benzoylamino]-2-methyl-phenyl}-amide; 2-(2-Methyl-6-morpholin-4-yl-pyrimidin-4-ylamino)-thiazole-5-carboxylic acid {5-[3-(4-ethyl-piperazin-1-yl)-5-trifluoromethyl-benzoylamino]-2-methyl-phenyl}-amide; 2-[6-(4-Ethyl-piperazin-1-yl)-2-methyl-pyrimidin-4-ylamino]-thiazole-5-carboxylic acid {5-[3-(4-ethyl-piperazin-1-yl)-5-trifluoromethyl-benzoylamino]-2-methyl-phenyl}-amide; 2-[6-(3-Diethylamino-propylamino)-2-methyl-pyrimidin-4-ylamino]-thiazole-5-carboxylic acid {5-[3-(4-ethyl-piperazin-1-yl)-5-trifluoromethyl-benzoylamino]-2-methyl-phenyl}-amide; 2-(2-Methyl-6-methylamino-pyrimidin-4-ylamino)-thiazole-5-carboxylic acid {5-[3-(4-ethyl-piperazin-1-yl)-5-trifluoromethyl-benzoylamino]-2-methyl-phenyl}-amide; 2-[6-(2-Hydroxy-ethylamino)-2-methyl-pyrimidin-4-ylamino]-thiazole-5-carboxylic acid {2-methyl-5-[3-(4-methyl-imidazol-1-yl)-5-trifluoromethyl-benzoylamino]-phenyl}-amide; 2-(6-Cyclopropylamino-2-methyl-pyrimidin-4-ylamino)-thiazole-5-carboxylic acid {2-methyl-5-[3-(4-methyl-imidazol-1-yl)-5-trifluoromethyl-benzoylamino]-phenyl}-amide; 2-(2-Methyl-6-morpholin-4-yl-pyrimidin-4-ylamino)-thiazole-5-carboxylic acid {2-methyl-5-[3-(4-methyl-imidazol-1-yl)-5-trifluoromethyl-benzoylamino]-phenyl}-amide; 2-[6-(4-Ethyl-piperazin-1-yl)-2-methyl-pyrimidin-4-ylamino]-thiazole-5-carboxylic acid {2-methyl-5-[3-(4-methyl-imidazol-1-yl)-5-trifluoromethyl-benzoylamino]-phenyl}-amide; 2-[6-(3-Diethylamino-propylamino)-2-methyl-pyrimidin-4-ylamino]-thiazole-5-carboxylic acid {2-methyl-5-[3-(4-methyl-imidazol-1-yl)-5-trifluoromethyl-benzoylamino]-phenyl}-amide; 2-Cyclopropylamino-thiazole-5-carboxylic acid {5-[4-(4-ethyl-piperazin-1-ylmethyl)-3-trifluoromethyl-phenylcarbamoyl]-2-methyl-phenyl}-amide; 2-Methylamino-thiazole-5-carboxylic acid {5-[4-(4-ethyl-piperazin-1-ylmethyl)-3-trifluoromethyl-phenylcarbamoyl]-2-methyl-phenyl}-amide; 2-Amino-thiazole-5-carboxylic acid {5-[4-(4-ethyl-piperazin-1-ylmethyl)-3-trifluoromethyl-phenylcarbamoyl]-2-methyl-phenyl}-amide; 2-(Pyridin-2-ylamino)-thiazole-5-carboxylic acid {5-[4-(4-ethyl-piperazin-1-ylmethyl)-3-trifluoromethyl-phenylcarbanoyl]-2-methyl-phenyl}-amide; 2-(Pyridin-2-ylamino)-thiazole-5-carboxylic acid [2-methyl-5-(4-morpholin-4-ylmethyl-3-trifluoromethyl-phenylcarbamoyl)-phenyl]-amide; 2-(Pyridin-2-ylamino)-thiazole-5-carboxylic acid [2-methyl-5-(4-piperazin-1-ylmethyl-3-trifluoromethyl-phenylcarbamoyl)-phenyl]-amide; 2-(Pyridin-2-ylamino)-thiazole-5-carboxylic acid {2-methyl-5-[4-(4-methyl-piperazin-1-ylmethyl)-3-trifluoromethyl-phenylcarbamoyl]-phenyl}-amide; 2-Cyclopropylamino-thiazole-5-carboxylic acid {2-methyl-5-[3-(4-methyl-imidazol-1-yl)-5-trifluoromethyl-phenylcarbamoyl]-phenyl}-amide; 2-Methylamino-thiazole-5-carboxylic acid {2-methyl-5-[4-(4-methyl-piperazin-1-ylmethyl)-3-trifluoromethyl-phenylcarbamoyl]-phenyl}-amide; 2-Cyclopropylamino-thiazole-5-carboxylic acid [2-methyl-5-(4-piperazin-1-ylmethyl-3-trifluoromethyl-phenylcarbamoyl)-phenyl]-amide; 2-Methylamino-thiazole-5-carboxylic acid [2-methyl-5-(4-piperazin-1-ylmethyl-3-trifluoromethyl-phenylcarbamoyl)-phenyl]-amide; 2-Cyclopropylamino-thiazole-5-carboxylic acid [2-methyl-5-(4-morpholin-4-ylmethyl-3-trifluoromethyl-phenylcarbamoyl)-phenyl]-amide; 2-Methylamino-thiazole-5-carboxylic acid [2-methyl-5-(4-morpholin-4-ylmethyl-3-trifluoromethyl-phenylcarbamoyl)-phenyl]-amide; 2-Cyclopropylamino-thiazole-5-carboxylic acid (5-{[1-tert-butyl-5-(4-methyl-piperazin-1-ylmethyl)-1H-pyrazole-3-carbonyl]-amino}-2-methyl-phenyl)-amide; 2-Cyclopropylamino-thiazole-5-carboxylic acid {2-methyl-5-[3-(4-methyl-piperazin-1-yl)-5-trifluoromethyl-benzoylamino]-phenyl}-amide; 2-Methylamino-thiazole-5-carboxylic acid {2-methyl-5-[3-(4-methyl-imidazol-1-yl)-5-trifluoromethyl-phenylcarbamoyl]-phenyl}-amide; 2-Cyclopropylamino-thiazole-5-carboxylic acid {2-methyl-5-[4-(4-methyl-piperazin-1-ylmethyl)-3-trifluoromethyl-phenylcarbamoyl]-phenyl}-amide; 2-Cyclopropylamino-thiazole-5-carboxylic acid {5-[3-(4-ethyl-piperazin-1-yl)-5-trifluoromethyl-benzoylamino]-2-methyl-phenyl}-amide; 2-Cyclopropylamino-thiazole-5-carboxylic acid {5-[4-(4-ethyl-piperazin-1-ylmethyl)-3-trifluoromethyl-benzoylamino]-2-methyl-phenyl}-amide; 2-Cyclopropylamino-thiazole-5-carboxylic acid {2-methyl-5-[3-(4-methyl-imidazol-1-yl)-5-trifluoromethyl-benzoylamino]-phenyl}-amide; 2-Cyclopropylamino-thiazole-5-carboxylic acid (5-{3-[4-(2-hydroxy-ethyl)-piperazin-1-yl]-5-trifluoromethyl-benzoylamino}-2-methyl-phenyl)-amide; 2-(2-Morpholin-4-yl-ethylamino)-thiazole-5-carboxylic acid {5-[(5-tert-butyl-thiophene-2-carbonyl)-amino]-2-methyl-phenyl}-amide; 2-(Pyridin-2-ylamino)-thiazole-5-carboxylic acid {5-[(5-tert-butyl-thiophene-2-carbonyl)-amino]-2-methyl-phenyl}-amide; 2-(Pyridin-2-ylamino)-thiazole-5-carboxylic acid {5-[(5-tert-butyl-2-methyl-2H-pyrazole-3-carbonyl)-amino]-2-methyl-phenyl}-amide; 2-{5-[4-(2-Hydroxy-ethyl)-piperazin-1-yl]-pyridin-2-ylamino}-thiazole-5-carboxylic acid [2-methyl-5-(3-trifluoromethyl-benzoylamino)-phenyl]-amide; 2-(Pyridin-2-ylamino)-thiazole-5-carboxylic acid (5-{3-[4-(2-hydroxy-ethyl)-piperazin-1-yl]-5-trifluoromethyl-benzoylamino}-2-methyl-phenyl)-amide; 2-(Pyridin-2-ylamino)-thiazole-5-carboxylic acid {5-[3-(4-ethyl-piperazin-1-yl)-5-trifluoromethyl-benzoylamino]-2-methyl-phenyl}-amide; 2-(Pyridin-2-ylamino)-thiazole-5-carboxylic acid [2-methyl-5-(3-trifluoromethyl-phenylcarbamoyl)-phenyl]-amide; 2-(Pyridin-3-ylamino)-thiazole-5-carboxylic acid [2-methyl-5-(3-trifluoromethyl-phenylcarbamoyl)-phenyl]-amide; 2-Cyclopropylamino-thiazole-5-carboxylic acid [2-methyl-5-(3-trifluoromethyl-phenylcarbamoyl)-phenyl]-amide; 2-(3-Imidazol-1-yl-propylamino)-thiazole-5-carboxylic acid [2-methyl-5-(3-trifluoromethyl-phenylcarbamoyl)-phenyl]-amide; 2-(2-Morpholin-4-yl-ethylamino)-thiazole-5-carboxylic acid {5-[(5-tert-butyl-2-methyl-2H-pyrazole-3-carbonyl)-amino]-2-methyl-phenyl}-amide; 2-(Pyridin-2-ylamino)-thiazole-5-carboxylic acid [5-(4-chloro-3-trifluoromethyl-benzoylamino)-2-methyl-phenyl]-amide; 2-(Pyridin-2-ylamino)-thiazole-5-carboxylic acid {5-[(1-tert-butyl-5-methyl-1H-pyrazole-3-carbonyl)-amino]-2-methyl-phenyl}-amide; 2-(Pyridin-2-ylamino)-thiazole-5-carboxylic acid {2-methyl-5-[3-(pyrrolidin-2-ylmethoxy)-5-trifluoromethyl-benzoylamino]-phenyl}-amide; 2-(Pyridin-2-ylamino)-thiazole-5-carboxylic acid {2-methyl-5-[3-(4-methyl-piperazin-1-yl)-5-trifluoromethyl-benzoylamino]-phenyl}-amide; 2-(Pyridin-2-ylamino)-thiazole-5-carboxylic acid {2-methyl-5-[3-(4-methyl-imidazol-1-yl)-5-trifluoromethyl-benzoylamino]-phenyl}-amide; 2-(6-Methyl-pyridin-3-ylamino)-thiazole-5-carboxylic acid [2-methyl-5-(3-trifluoromethyl-phenylcarbamoyl)-phenyl]-amide; 2-(2-Morpholin-4-yl-ethylamino)-thiazole-5-carboxylic acid [2-methyl-5-(3-trifluoromethyl-phenylcarbamoyl)-phenyl]-amide; 2-Isopropylamino-thiazole-5-carboxylic acid [2-methyl-5-(3-trifluoromethyl-phenylcarbamoyl)-phenyl]-amide; 2-[3-(4-Methyl-piperazin-1-yl)-propylamino]-thiazole-5-carboxylic acid [2-methyl-5-(3-trifluoromethyl-phenylcarbamoyl)-phenyl]-amide; 2-(Pyridin-2-ylamino)-thiazole-5-carboxylic acid [2-methyl-5-(4-piperazin-1-ylmethyl-3-trifluoromethyl-benzoylamino)-phenyl]-amide; 2-(Pyridin-2-ylamino)-thiazole-5-carboxylic acid {5-[4-(4-ethyl-piperazin-1-ylmethyl)-3-trifluoromethyl-benzoylamino]-2-methyl-phenyl}-amide; 2-Cyclopropylamino-thiazole-5-carboxylic acid [2-methyl-5-(4-morpholin-4-ylmethyl-3-trifluoromethyl-benzoylamino)-phenyl]-amide; 2-{6-[4-(2-Hydroxy-ethyl)-piperazin-1-yl]-2-methyl-pyrimidin-4-ylamino}-thiazole-5-carboxylic acid [2-methyl-5-(3-trifluoromethyl-benzoylamino)-phenyl]-amide; 2-[6-(4-Methyl-piperazin-1-yl)-pyrimidin-4-ylamino]-thiazole-5-carboxylic acid [2-methyl-5-(3-trifluoromethyl-benzoylamino)-phenyl]-amide; 2-{6-[4-(2-Hydroxy-ethyl)-piperazin-1-yl]-pyrimidin-4-ylamino}-thiazole-5-carboxylic acid [2-methyl-5-(3-trifluoromethyl-benzoylamino)-phenyl]-amide; 2-[2-Methyl-6-(4-methyl-piperazin-1-yl)-pyrimidin-4-ylamino]-thiazole-5-carboxylic acid [2-methyl-5-(3-trifluoromethyl-benzoylamino)-phenyl]-amide; and 2-{4-[4-(2-Hydroxy-ethyl)-piperazin-1-yl]-pyridin-2-ylamino}-thiazole-5-carboxylic acid [2-methyl-5-(3-trifluoromethyl-benzoylamino)-phenyl]-amide.

### Pharmacology and Utility

Compounds of the invention modulate the activity of kinases and, as such, are useful for treating diseases or disorders in which kinases, contribute to the pathology and/or symptomology of the disease. Examples of kinases that are inhibited by the compounds and compositions described herein and against which the methods described herein are useful include, but are not limited to, Abl, Bcr-Abl, FGFR3, PDGFRβ, Flt3 and b-Raf kinases.

Abelson tyrosine kinase (i.e. Abl, c-Abl) is involved in the regulation of the cell cycle, in the cellular response to genotoxic stress, and in the transmission of information about the cellular environment through integrin signaling. Overall, it appears that the Abl protein serves a complex role as a cellular module that integrates signals from various extracellular and intracellular sources and that influences decisions in regard to cell cycle and apoptosis. Abelson tyrosine kinase includes sub-types derivatives such as the chimeric fusion (oncoprotein) BCR-Abl with deregulated tyrosine kinase activity or the v-Abl. BCR-Abl is critical in the pathogenesis of 95% of chronic myelogenous leukemia (CML) and 10% of acute lymphocytic leukemia. STI-571 (Gleevec) is an inhibitor of the oncogenic BCR-Abl tyrosine kinase and is used for the treatment of chronic myeloid leukemia (CML). However, some patients in the blast crisis stage of CML are resistant to STI-571 due to mutations in the BCR-Abl kinase. Over 22 mutations have been reported to date with the most common being G250E, E255V, T315I, F317L and M351T.

Compounds of the present invention inhibit abl kinase, especially v-abl kinase. The compounds of the present invention also inhibit wild-type BCR-Abl kinase and mutations of BCR-Abl kinase and are thus suitable for the treatment of Bcr-abl-positive cancer and tumor diseases, such as leukemias (especially chronic myeloid leukemia and acute lymphoblastic leukemia, where especially apoptotic mechanisms of action are found), and also shows effects on the subgroup of leukemic stem cells as well as potential for the purification of these cells in vitro after removal of said cells (for example, bone marrow removal) and reimplantation of the cells once they have been cleared of cancer cells (for example, reimplantation of purified bone marrow cells).

PDGF (Platelet-derived Growth Factor) is a very commonly occurring growth factor, which plays an important role both in normal growth and also in pathological cell proliferation, such as is seen in carcinogenesis and in diseases of the smooth-muscle cells of blood vessels, for example in atherosclerosis and thrombosis. Compounds of the invention can inhibit PDGF receptor (PDGFR) activity and are, therefore, suitable for the treatment of tumor diseases, such as gliomas, sarcomas, prostate tumors, and tumors of the colon, breast, and ovary.

Compounds of the present invention, can be used not only as a tumor-inhibiting substance, for example in small cell lung cancer, but also as an agent to treat non-malignant proliferative disorders, such as atherosclerosis, thrombosis, psoriasis, scleroderma and fibrosis, as well as for the protection of stem cells, for example to combat the hemotoxic effect of chemotherapeutic agents, such as 5-fluoruracil, and in asthma. Compounds of the invention can especially be used for the treatment of diseases, which respond to an inhibition of the PDGF receptor kinase.

Compounds of the present invention show useful effects in the treatment of disorders arising as a result of transplantation, for example, allogenic transplantation, especially tissue rejection, such as especially obliterative bronchiolitis (OB), i.e. a chronic rejection of allogenic lung transplants. In contrast to patients without OB, those with OB often show an elevated PDGF concentration in bronchoalveolar lavage fluids.

Compounds of the present invention are also effective in diseases associated with vascular smooth-muscle cell migration and proliferation (where PDGF and PDGF-R often also play a role), such as restenosis and atherosclerosis. These effects and the consequences thereof for the proliferation or migration of vascular smooth-muscle cells in vitro and in vivo can be demonstrated by administration of the compounds of the present invention, and also by investigating its effect on the thickening of the vascular intima following mechanical injury in vivo.

Certain abnormal proliferative conditions are believed to be associated with raf expression and are, therefore, believed to be responsive to inhibition of raf expression. Abnormally high levels of expression of the raf protein are also implicated in transformation and abnormal cell proliferation. These abnormal proliferative conditions are also believed to be responsive to inhibition of raf expression. For example, expression of the c-raf protein is believed to play a role in abnormal cell proliferation since it has been reported that 60% of all lung carcinoma cell lines express unusually high levels of c-raf mRNA and protein. Further examples of abnormal proliferative conditions are hyper-proliferative disorders such as cancers, tumors, hyperplasia, pulmonary fibrosis, angiogenesis, psoriasis, atherosclerosis and smooth muscle cell proliferation in the blood vessels, such as stenosis or restenosis following angioplasty. The cellular signaling pathway of which raf is a part has also been implicated in inflammatory disorders characterized by T-cell proliferation (T-cell activation and growth), such as tissue graft rejection, endotoxin shock, and glomerular nephritis, for example.

Flt3 is a member of the type III receptor tyrosine kinase (RTK) family. Flt3 (fins-like tyrosine kinase) is also known as FLk-2 (fetal liver kinase 2). Aberrant expression of the Flt3 gene has been documented in both adult and childhood leukemias including acute myeloid leukemia (AML), AML with trilineage myelodysplasia (AML/TMDS), acute lymphoblastic leukemia (ALL), and myelodysplastic syndrome (MDS). Activating mutations of the Flt3 receptor have been found in about 35% of patients with acute myeloblastic leukemia (AML), and are associated with a poor prognosis. The most common mutation involves in-frame duplication within the juxtamembrane domain, with an additional 5-10% of patients having a point mutation at asparagine 835. Both of these mutations are associated with constitutive activation of the tyrosine kinase activity of Flt3, and result in proliferation and viability signals in the absence of ligand. Patients expressing the mutant form of the receptor have been shown to have a decreased chance for cure. Thus, there is accumulating evidence for a role for hyper-activated (mutated) Flt3 kinase activity in human leukemias and myelodysplastic syndrome. This has prompted the applicant to search for new inhibitors of the Flt3 receptor as a possible therapeutic approach in these patients, for whom current drug therapies offer little utility, and for such patients who have previously failed current available drug therapies and/or stem cell transplantation therapies.

Leukemias generally result from an acquired (not inherited) genetic injury to the DNA of immature hematopoietic cells in the bone marrow, lymph nodes, spleen, or other organs of the blood and immune system. The effects are: the accelerated growth and blockage in the maturation of cells, resulting in the accumulation of cells called “leukemic blasts”, which do not function as normal blood cells; and a failure to produce normal marrow cells, leading to a deficiency of red cells (anemia), platelets and normal white cells. Blast cells are normally produced by bone marrow and usually develop into mature blood cells, comprising about 1 percent of all marrow cells. In leukemia, the blasts do not mature properly and accumulate in the bone marrow. In acute myeloid leukemia (AML), these are called myeloblasts while in acute lymphoblastic leukemia (ALL) they are known as lymphoblasts. Another leukemia is mixed-lineage leukemia (MLL).

The term “AML with trilineage myelodysplasia (AML/TMDS)” relates to an uncommon form of leukemia characterized by a dyshematopoietic picture accompanying the acute leukemia, a poor response to induction chemotherapy, and a tendency to relapse with pure myelodysplastic syndrome.

The term “Myelodysplastic Syndrome (MDS)” relates to a group of blood disorders in which the bone marrow stops functioning normally, resulting in a deficiency in the number of healthy blood cells. Compared with leukemia, in which one type of blood cell is produced in large numbers, any and sometimes all types of blood cells are affected in MDS. At least 10,000 new cases occur annually in the United States. Up to one third of patients diagnosed with MDS go on to develop acute myeloid leukemia. For this reason the disease is sometimes referred to as preleukemia. Myelodysplastic syndrome is sometimes also called myelodysplasia dysmyelopoiesis or oligoblastic leukemia. MDS is also referred to as smoldering leukemia when high numbers of blast cells remain in the marrow.

Myelodysplastic syndrome, like leukemia, results from a genetic injury to the DNA of a single cell in the bone marrow. Certain abnormalities in chromosomes are present in MDS patients. These abnormalities are called translocations, which occur when a part of one chromosome breaks off and becomes attached to a broken part of a different chromosome. The same defects are frequently found in acute myeloid leukemia. However, MDS differs from leukemia because all of the patient's blood cells are abnormal and all are derived from the same damaged stem cell. In leukemia patients, the bone marrow contains a mixture of diseased and healthy blood cells.

AML and advanced myelodysplastic syndromes are currently treated with high doses of cytotoxic chemotherapy drugs such cytosine arabinoside and daunorubicin. This type of treatment induces about 70% of patients to enter a hematological remission. However, more than half of the patients that enter remission will later relapse despite administration of chemotherapy over long periods of time. Almost all of the patients who either fail to enter remission initially, or relapse later after obtaining remission, will ultimately die because of leukemia. Bone marrow transplantation can cure up to 50-60% of patients who undergo the procedure, but only about one third of all patients with AML or MDS are eligible to receive a transplant. New and effective drugs are urgently needed to treat the patients who fail to enter remission with standard therapies, patients who later relapse, and patients that are not eligible for stem cell transplantation. Further, an effective new drug could be added to standard therapy with the reasonable expectation that it will result in improved induction chemotherapy for all patients.

FGFR3 is part of a family of structurally related tyrosine kinase receptors encoded by 4 different genes. Specific point mutations in different domains of the FGFR3 gene lead to constitutive activation of the receptor and are associated with autosomal dominant skeletal disorders, multiple myeloma, and a large proportion of bladder and cervical cancer (Cappellen, et al, Nature, vol. 23). Activating mutations placed in the mouse FGFR3 gene and the targeting of activated FGFR3 to growth plate cartilage in mice result in dwarfism. Analogous to our concept, targeted disruption of FGFR3 in mice results in the overgrowth of long bones and vertebrae. In addition, 20-25% of multiple myeloma cells contain a t(4; 14)(p16.3; q32.3) chromosomal translocation with breakpoints on 4p16 located 50-100 kb centromeric to FGFR3. In rare cases of multiple myeloma, activating mutations of FGFR3 previously seen in skeletal disorders have been found and are always accompanied by this chromosomal translocation. Recently, FGFR3 missense somatic mutations (R248C, S249C, G372C, and K652E) have been identified in a large proportion of bladder cancer cells and in some cervical cancer cells, and these in fact are identical to the germinal activating mutations that cause thanatophoric dysplasia, a form of dwarfism lethal in the neonatal period. Compounds of the invention can have therapeutic utility for multiple myeloma by being more effective than current treatment, for bladder cancer by avoiding life-altering cystectomy, and for cervical cancer in those patients who wish to preserve future fertility.

In accordance with the foregoing, the present invention further provides a method for preventing or treating any of the diseases or disorders described above in a subject in need of such treatment, which method comprises administering to said subject a therapeutically effective amount (See, “Administration and Pharmaceutical Compositions”, infra) of a compound of Formula I or a pharmaceutically acceptable salt thereof. For any of the above uses, the required dosage will vary depending on the mode of administration, the particular condition to be treated and the effect desired.

### Administration and Pharmaceutical Compositions

In general, compounds of the invention will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors. In general, satisfactory results are indicated to be obtained systemically at daily dosages of from about 0.03 to 2.5 mg/kg per body weight. An indicated daily dosage in the larger mammal, e.g. humans, is in the range from about 0.5 mg to about 100 mg, conveniently administered, e.g. in divided doses up to four times a day or in retard form. Suitable unit dosage forms for oral administration comprise from ca. 1 to 50 mg active ingredient.

Compounds of the invention can be administered as pharmaceutical compositions by any conventional route, in particular enterally, e.g., orally, e.g., in the form of tablets or capsules, or parenterally, e.g., in the form of injectable solutions or suspensions, topically, e.g., in the form of lotions, gels, ointments or creams, or in a nasal or suppository form. Pharmaceutical compositions comprising a compound of the present invention in free form or in a pharmaceutically acceptable salt form in association with at least one pharmaceutically acceptable carrier or diluent can be manufactured in a conventional manner by mixing, granulating or coating methods. For example, oral compositions can be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrrolidone; if desired d) disintegrants, e.g., starches, agar, alginic acid or its sodium salt, or effervescent mixtures; and/or e) absorbents, colorants, flavors and sweeteners. Injectable compositions can be aqueous isotonic solutions or suspensions, and suppositories can be prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Suitable formulations for transdermal applications include an effective amount of a compound of the present invention with a carrier. A carrier can include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used. Suitable formulations for topical application, e.g., to the skin and eyes, are preferably aqueous solutions, ointments, creams or gels well-known in the art. Such may contain solubilizers, stabilizers, tonicity enhancing agents, buffers and preservatives.

Compounds of the invention can be administered in therapeutically effective amounts in combination with one or more therapeutic agents (pharmaceutical combinations). For example, synergistic effects can occur with other immunomodulatory or anti-inflammatory substances, for example when used in combination with cyclosporin, rapamycin, or ascomycin, or immunosuppressant analogues thereof, for example cyclosporin A (CsA), cyclosporin G, FK-506, rapamycin, or comparable compounds, corticosteroids, cyclophosphamide, azathioprine, methotrexate, brequinar, leflunomide, mizoribine, mycophenolic acid, mycophenolate mofetil, 15-deoxyspergualin, immunosuppressant antibodies, especially monoclonal antibodies for leukocyte receptors, for example MHC, CD2, CD3, CD4, CD7, CD25, CD28, B7, CD45, CD58 or their ligands, or other immunomodulatory compounds, such as CTLA41g. Where the compounds of the invention are administered in conjunction with other therapies, dosages of the co-administered compounds will of course vary depending on the type of co-drug employed, on the specific drug employed, on the condition being treated and so forth.

The invention also provides for a pharmaceutical combinations, e.g. a kit, comprising a) a first agent which is a compound of the invention as disclosed herein, in free form or in pharmaceutically acceptable salt form, and b) at least one co-agent. The kit can comprise instructions for its administration.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

The term “pharmaceutical combination” as used herein means a product that results from the mixing or combining of more than one active ingredient and includes both fixed and non-fixed combinations of the active ingredients. The term “fixed combination” means that the active ingredients, e.g. a compound of Formula I and a co-agent, are both administered to a patient simultaneously in the form of a single entity or dosage. The term “non-fixed combination” means that the active ingredients, e.g. a compound of Formula I and a co-agent, are both administered to a patient as separate entities either simultaneously, concurrently or sequentially with no specific time limits, wherein such administration provides therapeutically effective levels of the 2 compounds in the body of the patient. The latter also applies to cocktail therapy, e.g. the administration of 3 or more active ingredients.

### Processes for Making Compounds of the Invention

The present invention also includes processes for the preparation of compounds of the invention. In the reactions described, it can be necessary to protect reactive functional groups, for example hydroxy, amino, imino, thio or carboxy groups, where these are desired in the final product, to avoid their unwanted participation in the reactions. Conventional protecting groups can be used in accordance with standard practice, for example, see T. W. Greene and P. G. M. Wuts in “Protective Groups in Organic Chemistry”, John Wiley and Sons, 1991.

Compounds of Formula I can be prepared by proceeding as in the following Reaction Scheme I:

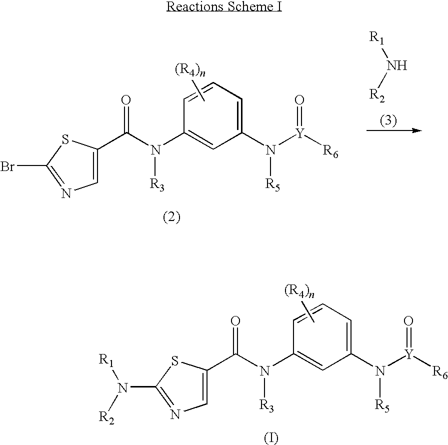

in which n, R₁, R₂, R₃, R₄, R₅ and R₆ are defined in the Summary of the Invention. A compound of Formula I can be prepared by reacting a compound of formula 2 with a compound of formula 3 in the presence of a suitable solvent (e.g., 1,3-dimethyl-2-imidazolidone, or the like). The reaction proceeds in a temperature range of about 50° C. to about 120° C. and can take up to 12 hours to complete.

Detailed examples of the synthesis of a compound of Formula I can be found in the Examples, infra.

### Additional Processes for Making Compounds of the Invention

A compound of the invention can be prepared as a pharmaceutically acceptable acid addition salt by reacting the free base form of the compound with a pharmaceutically acceptable inorganic or organic acid. Alternatively, a pharmaceutically acceptable base addition salt of a compound of the invention can be prepared by reacting the free acid form of the compound with a pharmaceutically acceptable inorganic or organic base.

Alternatively, the salt forms of the compounds of the invention can be prepared using salts of the starting materials or intermediates.

The free acid or free base forms of the compounds of the invention can be prepared from the corresponding base addition salt or acid addition salt from, respectively. For example a compound of the invention in an acid addition salt form can be converted to the corresponding free base by treating with a suitable base (e.g., ammonium hydroxide solution, sodium hydroxide, and the like). A compound of the invention in a base addition salt form can be converted to the corresponding free acid by treating with a suitable acid (e.g., hydrochloric acid, etc.).

Compounds of the invention in unoxidized form can be prepared from N-oxides of compounds of the invention by treating with a reducing agent (e.g., sulfur, sulfur dioxide, triphenyl phosphine, lithium borohydride, sodium borohydride, phosphorus trichloride, tribromide, or the like) in a suitable inert organic solvent (e.g. acetonitrile, ethanol, aqueous dioxane, or the like) at 0 to 80° C.

Prodrug derivatives of the compounds of the invention can be prepared by methods known to those of ordinary skill in the art (e.g., for further details see Saulnier et al., (1994), Bioorganic and Medicinal Chemistry Letters, Vol. 4, p. 1985). For example, appropriate prodrugs can be prepared by reacting a non-derivatized compound of the invention with a suitable carbamylating agent (e.g., 1,1-acyloxyalkylcarbanochloridate, para-nitrophenyl carbonate, or the like).

Protected derivatives of the compounds of the invention can be made by means known to those of ordinary skill in the art. A detailed description of techniques applicable to the creation of protecting groups and their removal can be found in T. W. Greene, “Protecting Groups in Organic Chemistry”, 3^{rd} edition, John Wiley and Sons, Inc., 1999.

Compounds of the present invention can be conveniently prepared, or formed during the process of the invention, as solvates (e.g., hydrates). Hydrates of compounds of the present invention can be conveniently prepared by recrystallization from an aqueous/organic solvent mixture, using organic solvents such as dioxin, tetrahydrofuran or methanol.

Compounds of the invention can be prepared as their individual stereoisomers by reacting a racemic mixture of the compound with an optically active resolving agent to form a pair of diastereoisomeric compounds, separating the diastereomers and recovering the optically pure enantiomers. While resolution of enantiomers can be carried out using covalent diastereomeric derivatives of the compounds of the invention, dissociable complexes are preferred (e.g., crystalline diastereomeric salts). Diastereomers have distinct physical properties (e.g., melting points, boiling points, solubilities, reactivity, etc.) and can be readily separated by taking advantage of these dissimilarities. The diastereomers can be separated by chromatography, or preferably, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques, Andre Collet, Samuel H. Wilen, “Enantiomers, Racemates and Resolutions”, John Wiley And Sons, Inc., 1981.

In summary, the compounds of Formula I can be made by a process, which involves:

(a) that of reaction schemes I; and

(b) optionally converting a compound of the invention into a pharmaceutically acceptable salt;

(c) optionally converting a salt form of a compound of the invention to a non-salt form;

(d) optionally converting an unoxidized form of a compound of the invention into a pharmaceutically acceptable N-oxide;

(e) optionally converting an N-oxide form of a compound of the invention to its unoxidized form;

(f) optionally resolving an individual isomer of a compound of the invention from a mixture of isomers;

(g) optionally converting a non-derivatized compound of the invention into a pharmaceutically acceptable prodrug derivative; and

(h) optionally converting a prodrug derivative of a compound of the invention to its non-derivatized form.

Insofar as the production of the starting materials is not particularly described, the compounds are known or can be prepared analogously to methods known in the art or as disclosed in the Examples hereinafter.

One of skill in the art will appreciate that the above transformations are only representative of methods for preparation of the compounds of the present invention, and that other well known methods can similarly be used.

### EXAMPLES

The present invention is further exemplified, but not limited, by the following examples that illustrate the preparation of compounds of Formula I according to the invention.

### Example 1

### 2-(3-Diethylaminopropylamino)-thiazole-5-carboxylic acid [2-methyl-5-(3-trifluoromethyl-benzoylamino)-phenyl]-amide

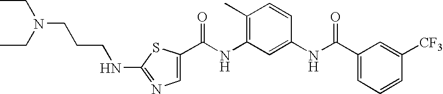

To a stirred solution of 4-methyl-3-nitroaniline (1.00 g, 6.57 mmol) and triethylamine (1.10 mL, 7.89 mmol) at 0° C. is added 3-trifluoromethylbenzoyl chloride (4.90 g, 31.0 mmol) and the mixture is stirred for 1 hour at room temperature. The reaction mixture is diluted with EtOAc and washed with saturated aqueous sodium bicarbonate solution. The organic layer is dried over MgSO₄ and concentrated in reduced pressure to give a crude product. The crude product is dissolved in MeOH and 10% Pd/C is added to the solution. The reaction mixture is stirred for 12 hours at room temperature under hydrogen. The reaction mixture is filtered on Celite plate and the filtrate is concentrated under reduced pressure to give N-(3-amino-4-methylphenyl)-3-trifluoromethyl-benzamide as a dark-gray solid.

To a stirred solution of N-(4-methyl-3-nitrophenyl)-3-trifluoromethylbenzamide (250 mg, 0.85 mmol), 2-bromothiazole-5-carboxylic acid (177 mg, 0.85 mmol), and diisopropylethyl-amine (0.59 mL, 3.4 mmol) in DMF is added O-(7-azabenzotriazol-1-yl)-N,N,N′,N′-tetramethyluronium hexafluorophosphate (355 mg, 0.93 mmol), and the mixture is stirred for 12 hours at room temperature. The reaction mixture is diluted with EtOAc and washed with 10% aqueous sodium thiosulfate solution. The organic layer is dried over MgSO₄ and concentrated in reduced pressure. The crude product is purified by preparative HPLC to give 2-bromothiazole-5-carboxylic acid [2-methyl-5-(3-trifluoromethyl-benzoylamino)-phenyl]-amide as a brownish solid.

2-Bromothiazole-5-carboxylic acid [2-methyl-5-(3-trifluoromethylbenzoyl-amino)-phenyl]-amide (25 mg, 52 μmol) is dissolved in 3-(diethylamino)-propylamine and the mixture is stirred for 4 hours at 80° C. The crude product is diluted with DMSO (1 mL) and purified by preparative HPLC to give 2-(3-diethylaminopropylamino)-thiazole-5-carboxylic acid [2-methyl-5-(3-trifluoromethyl-benzoylamino)-phenyl]-amide in a TFA salt form: ¹H NMR 400 MHz (DMSO-d₆) δ 9.67 (s, 1H), 9.43 (br, 1H), 8.35 (t, 1H), 8.29 (s, 1H), 8.26 (d, 1H), 7.96 (d, 1H), 7.94 (s, 1H), 7.80 (d, 1H), 7.58 (d, 1H), 7.25 (d, 1H), 3.35 (q, 2H), 2.89 (m, 6H), 2.19 (s, 3H), 1.93 (m, 2H), 1.20 (t, 6H); MS m/z 534.4 (M+1).

### Example 2

### 2-{6-[4-(2-Hydroxyethyl)-piperazin-1-yl]-2-methylpyrimidin-4-ylamino}-thiazole-5-carboxylic acid [2-methyl-5-(3-trifluoromethylbenzoylamino)-phenyl]-amide

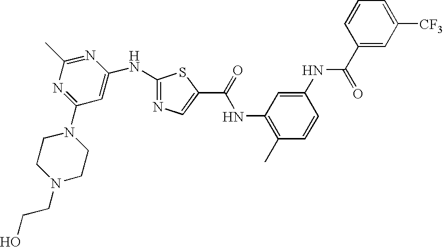

To a suspension of methyl 2-aminothiazole-5-carboxylate (4.90 g, 31.0 mmol) and NaH (60% dispersion in mineral oil, 1.36 g, 34.1 mmol) in DMF at 0° C. is added 4,6-dichloro-2-methyl-pyrimidine (5.05 g, 31.0 mmol) in DMF and the mixture is stirred for 2 hours at room temperature. The reaction mixture is diluted with EtOAc and washed with 10% aqueous sodium thiosulfate solution. The organic layer is dried over MgSO₄, and concentrated in reduced pressure. The crude product is crystallized from MeOH to give methyl 2-(6-chloro-2-methyl-pyrimidin-4-ylamino)-thiazole-5-carboxylate as a white solid.

To a stirred solution of methyl 2-(6-chloro-2-methyl-pyrimidin-4-ylamino)-thiazole-5-carboxylate (3.97 g, 14.0 mmol) in MeOH is added 4 N NaOH (15 mL) and the mixture is stirred for 12 hours at 60° C. The reaction mixture is neutralized with 1 N HCl and the resulting precipitate is filtered and washed with MeOH to give 2-(6-chloro-2-methyl-pyrimidin-4-ylamino)-thiazole-5-carboxylic acid in a white solid.

To a solution of 2-(6-chloro-2-methyl-pyrimidin-4-ylamino)-thiazole-5-carboxylic acid (230 mg, 0.85 mmol), N-(3-Amino-4-methyl-phenyl)-3-trifluoromethylbenzamide (250 mg, 0.85 mmol), and diisopropylethylamine (0.59 mL, 3.4 mmol) in DMF is added O-(7-azabenzotriazol-1-yl)-N,N,N′,N′-tetramethyluronium hexafluorophosphate (355 mg, 0.93 mmol), and the mixture is stirred for 12 hours at room temperature. The reaction mixture is diluted with EtOAc and washed with 10% aqueous sodium thiosulfate solution. The organic layer is dried over MgSO₄ and concentrated in reduced pressure. The crude product is purified by preparative HPLC to give 2-(6-chloro-2-methyl-pyrimidin-4-ylamino)-thiazole-5-carboxylic acid [2-methyl-5-(3-trifluoromethyl-benzoylamino)-phenyl]-amide as a white solid.

To a stirred solution of 2-(6-chloro-2-methyl-pyrimidin-4-ylamino)-thiazole-5-carboxylic acid [2-methyl-5-(3-trifluoromethyl-benzoylamino)-phenyl]-amide (25 mg, 46 μmol) in 1,3-dimethyl-2-imidazolidinone (0.2 mL) is added excess 2-piperazin-1-yl-ethanol (100 mg) in 1,3-dimethyl-2-imidazolidinone (0.2 mL) and the mixture is stirred for 4 hours at 60° C. The crude product is diluted with DMSO (1 mL) and purified by preparative HPLC to give 2-{6-[4-(2-Hydroxyethyl)-piperazin-1-yl]-2-methylpyrimidin-4-ylamino}-thiazole-5-carboxylic acid [2-methyl-5-(3-trifluoromethylbenzoylamino)-phenyl]-amide in a TFA salt form: ¹H NMR 400 MHz (MeOH-d₄) δ 8.26 (s, 1H), 8.20 (d, 1H), 8.15 (s, 1H), 7.90 (d, 1H), 7.83 (s, 1H), 7.74 (t, 1H), 7.55 (d, 1H), 7.31 (d, 1H), 6.20 (br, 1H), 3.93 (dd, 2H), 3.50 (br, 8H), 3.35 (dd, 2H), 2.53 (s, 3H), 2.31 (s, 3H); MS m/z 641.5 (M+1).

### Example 3

### 2-(2-Hydroxy-ethylamino)-thiazole-5-carboxylic Acid {5-[3-(4-ethyl-piperazin-1-yl)-5-trifluoromethyl-benzoylamino]-2-methyl-phenyl}-amide

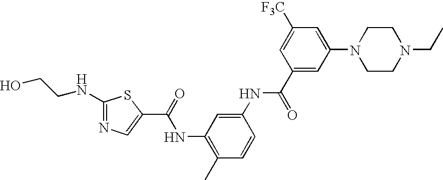

To a stirred solution of 4-methyl-3-nitroaniline (259 mg, 1.7 mmol), 3-(4-ethyl-piperazin-1-yl)-5-trifluoromethyl-benzoic acid (514 mg, 1.7 mmol), and diisopropylethyl-amine (1.19 mL, 6.8 mmol) in DMF is added O-(7-azabenzotriazol-1-yl)-N,N,N′, N′-tetramethyluronium hexafluorophosphate (710 mg, 1.9 mmol), and the mixture is stirred for 12 hours at room temperature. The reaction mixture is diluted with EtOAc and washed with 10% aqueous sodium thiosulfate solution. The organic layer is dried over MgSO₄ and concentrated under reduced pressure to give a crude product. The crude product is dissolved in MeOH and 10% Pd/C is added to the solution. The reaction mixture is stirred for 12 hours at room temperature under hydrogen. The reaction mixture is filtered on Celite plate and the filtrate is concentrated under reduced pressure to give N-(3-Amino-4-methylphenyl)-3-(4-ethyl-piperazin-1-yl)-5-trifluoromethylbenzamide.

To a stirred solution of N-(3-Amino-4-methylphenyl)-3-(4-ethyl-piperazin-1-yl)-5-trifluoromethylbenzamide (345 mg, 0.85 mmol), 2-bromothiazole-5-carboxylic acid (177 mg, 0.85 mmol), and diisopropylethyl-amine (0.59 mL, 3.4 mmol) in DMF is added 0-(7-azabenzotriazol-1-yl)-N,N,N′,N′-tetramethyluronium hexafluorophosphate (355 mg, 0.93 mmol), and the mixture is stirred for 12 hours at room temperature. The reaction mixture is diluted with EtOAc and washed with 10% aqueous sodium thiosulfate solution. The organic layer is dried over MgSO₄ and concentrated under reduced pressure. The crude product is purified by preparative HPLC to give 2-bromothiazole-5-carboxylic acid {5-[3-(4-ethyl-piperazin-1-yl)-5-trifluoromethylbenzoylamino]-2-methylphenyl}-amide as a brownish solid.

2-Bromothiazole-5-carboxylic acid {5-[3-(4-ethyl-piperazin-1-yl)-5-trifluoromethylbenzoylamino]-2-methylphenyl}-amide (25 mg, 42 μmol) is dissolved in ethanolamine and the mixture is stirred for 4 hours at 80° C. The crude product is diluted with DMSO (1 mL) and purified by preparative HPLC to give 2-(2-hydroxy-ethylamino)-thiazole-5-carboxylic acid {5-[3-(4-ethyl-piperazin-1-yl)-5-trifluoromethyl-benzoylamino]-2-methyl-phenyl}-amide in a TFA salt form: ¹H NMR 400 MHz (MeOH-d₄) δ 7.87 (s, 1H), 7.77 (s, 1H), 7.75 (s, 1H), 7.71 (s, 1H), 7.51 (d, 1H), 7.46 (s, 1H), 7.24 (d, 1H), 4.50 (br, 2H), 3.72 (m, 2H), 3.68 (br, 2H), 3.45 (m, 2H), 3.22 (br, 6H), 2.23 (s, 3H), 1.38 (t, 3H); MS m/z 577.5 (M+1).

By repeating the procedures described in the above examples, using appropriate starting materials, the following compounds of Formula I, as identified in Table 1, are obtained.

TABLE 1

| | | Physical Data |
|---|---|---|
| | | ¹H NMR 400 MHz |
| Compound | | (DMSO-d₆) and/or MS |
| Number | Structure | (m/z) |
| 1 | 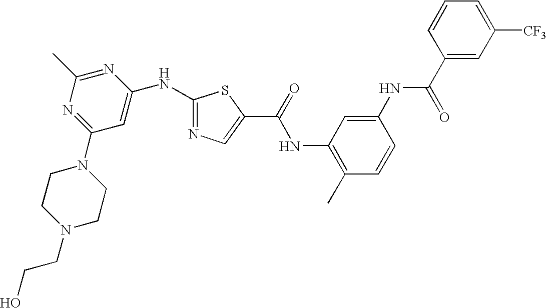 | ¹H NMR 400 MHz(MeOH-d₄) δ 8.26 (s, 1 H),8.20 (d, 1 H), 8.15 (s, 1 H),7.90 (d, 1 H), 7.83 (s, 1 H),7.74 (t, 1 H), 7.55 (d, 1 H),7.31 (d, 1 H), 6.20 (br, 1 H),3.93 (dd, 2 H), 3.50 (br,8 H), 3.35 (dd, 2 H), 2.53(s, 3 H), 2.31 (s, 3 H); MSm/z 641.5 (M + 1). |
| 2 | 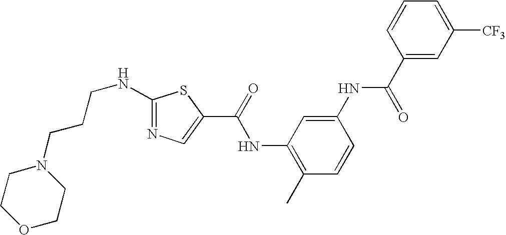 | ¹H NMR 400 MHz(DMSO-d₆) δ 9.87 (br,1 H), 9.66 (s, 1 H), 8.33 (t,1 H), 8.28 (s, 1 H), 8.25 (d,1 H), 7.94 (d, 1 H), 7.93 (s,1 H), 7.78 (d, 1 H), 7.54 (d,1 H), 7.22 (d, 1 H), 3.39 (br,4 H), 2.95 (m, 2 H), 2.67(br, 6 H), 2.17 (s, 3 H), 1.95(m, 2 H); MS m/z 548.4(M + 1). |
| 3 | 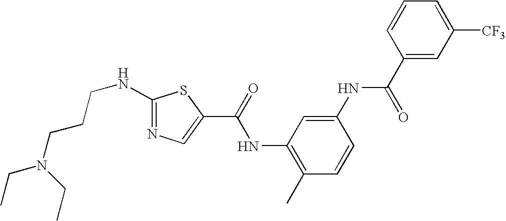 | ¹H NMR 400 MHz(DMSO-d₆) δ 9.67 (s, 1 H),9.43 (br, 1 H), 8.35 (t, 1 H),8.29 (s, 1 H), 8.26 (d, 1 H),7.96 (d, 1 H), 7.94 (s, 1 H),7.80 (d, 1 H), 7.58 (d, 1 H),7.25 (d, 1 H), 3.35 (q, 2 H),2.89 (m, 6 H), 2.19 (s, 3 H),1.93 (m, 2 H), 1.20 (t, 6 H);MS m/z 534.4 (M + 1). |
| 4 | 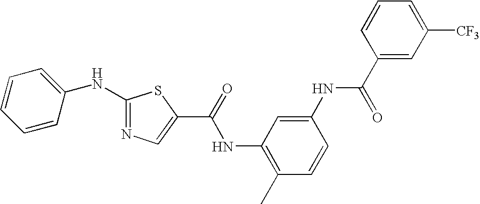 | ¹H NMR 400 MHz(DMSO-d₆) δ 9.86 (s, 1 H),8.21 (s, 1 H), 8.19 (d, 1 H),8.12 (s, 1 H), 8.01 (d, 1 H),7.82 (m, 2 H), 7.67 (m,3 H), 7.38 (m, 2 H), 7.32 (d,1 H), 7.05 (t, 1 H), 2.22 (s,3 H); MS m/z 497.3(M + 1). |
| 5 | 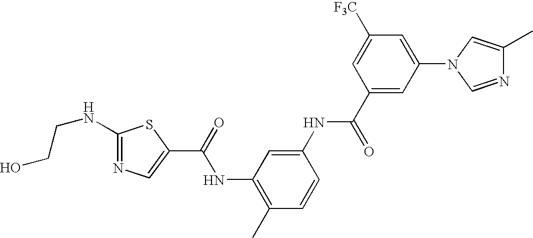 | ¹H NMR 400 MHz(MeOH-d₄) δ 9.56 (s, 1 H),8.68 (s, 1 H), 8.57 (s, 1 H),8.42 (s, 1 H), 8.06 (s, 1 H),8.01 (s, 1 H), 7.90 (s, 1 H),7.68 (d, 1 H), 7.39 (d, 1 H),3.86 (m, 2 H), 3.60 (m,2 H), 2.56 (s, 3 H), 2.38 (s,3 H); MS m/z 545.4(M + 1). |
| 6 | 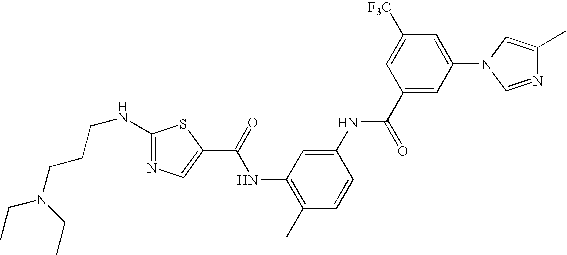 | ¹H NMR 400 MHz(DMSO-d₆) δ 9.59 (s, 1 H),8.45 (s, 1 H), 8.40 (s, 1 H),8.24 (s, 1 H), 8.17 (s, 1 H),7.92 (s, 1 H), 7.78 (s, 1 H),7.71 (s, 1 H), 7.59 (d, 1 H),7.24 (d, 1 H), 3.29 (m, 2 H),2.42 (m, 6 H), 2.21 (s, 3 H),2.20 (s, 3 H), 1.68 (m, 2 H),0.92 (t, 6 H); MS m/z614.5 (M + 1). |
| 7 | 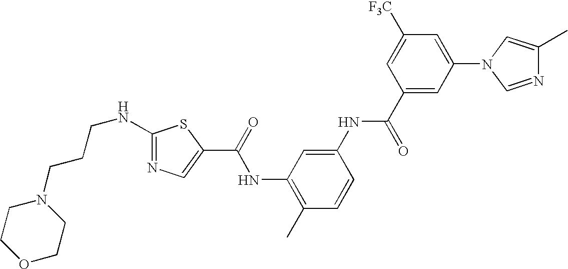 | ¹H NMR 400 MHz(DMSO-d₆) δ 9.59 (s, 1 H),8.43 (s, 1 H), 8.42 (s, 1 H),8.24 (s, 1 H), 8.16 (s, 1 H),7.92 (s, 1 H), 7.78 (s, 1 H),7.71 (s, 1 H), 7.59 (d, 1 H),7.24 (d, 1 H), 3.57 (m, 4 H),3.30 (m, 2 H), 2.31 (m,6 H), 2.21 (s, 3 H), 2.19 (s,3 H), 1.70 (m, 2 H); MS m/z628.4 (M + 1). |
| 8 | 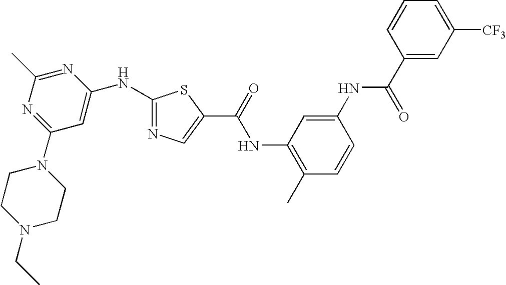 | ¹H NMR 400 MHz(MeOH-d₄) δ 8.26 (s, 1 H),8.21 (d, 1 H), 8.15 (s, 1 H),7.91 (d, 1 H), 7.82 (s, 1 H),7.73 (t, 1 H), 7.52 (d, 1 H),7.31 (d, 1 H), 6.20 (br, 1 H),3.60 (br, 4 H), 3.26 (q, 2 H),3.15 (br, 4 H), 2.54 (s, 3 H),2.30 (s, 3 H), 1.38 (t, 3 H);MS m/z 625.5 (M + 1). |
| 9 | 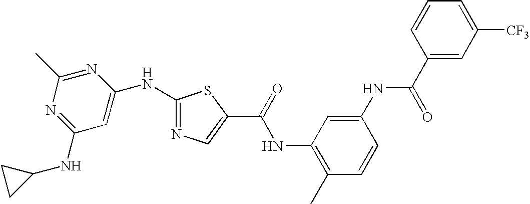 | ¹H NMR 400 MHz(DMSO-d₆) δ 9.85 (s, 1 H),8.28 (m, 3 H), 7.95 (d, 1 H),7.85 (s, 1 H), 7.80 (t, 1 H),7.59 (d, 1 H), 7.26 (d, 1 H),6.25 (br, 1 H), 2.58 (br,1 H), 2.45 (s, 3 H), 2.21 (s,3 H), 0.80 (m, 2 H), 0.54(m, 2 H); MS m/z 568.2(M + 1). |
| 10 | 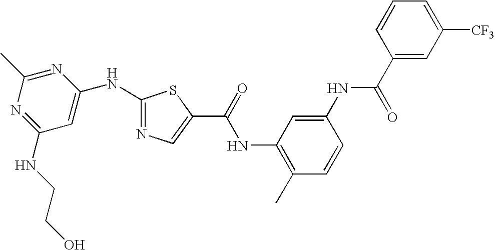 | ¹H NMR 400 MHz(MeOH-d₄) δ 8.16 (s, 1 H),8.10 (d, 1 H), 8.08 (s, 1 H),7.79 (d, 1 H), 7.71 (s, 1 H),7.62 (t, 1 H), 7.43 (d, 1 H),7.20 (d, 1 H), 6.19 (br, 1 H),3.68 (br, 2 H), 3.40 (br,2 H), 2.51 (s, 3 H), 2.20 (s,3 H); MS m/z 572.1(M + 1). |
| 11 | 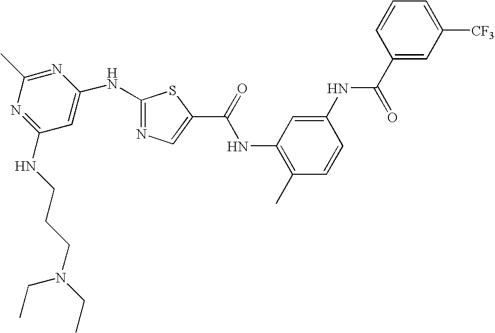 | ¹H NMR 400 MHz(MeOH-d₄) δ 8.43 (s, 1 H),8.36 (d, 1 H), 8.32 (s, 1 H),8.05 (d, 1 H), 7.98 (s, 1 H),7.88 (t, 1 H), 7.69 (d, 1 H),7.43 (d, 1 H), 6.31 (br, 1 H),3.65 (br, 2 H), 3.40 (m,6 H), 2.72 (s, 3 H), 2.42 (s,3 H), 2.21 (br, 2 H), 1.46 (t,3 H); MS m/z 641.2(M + 1). |
| 12 | 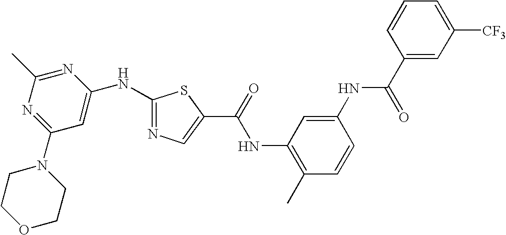 | ¹H NMR 400 MHz(DMSO-d₆) δ 9.79 (s, 1 H),8.30 (s, 1 H), 8.26 (d, 1 H),8.20 (s, 1 H), 7.96 (d, 1 H),7.82 (s, 1 H), 7.78 (t, 1 H),7.59 (d, 1 H), 7.24 (d, 1 H),6.06 (s, 1 H), 3.68 (t, 4 H),3.49 (t, 4 H), 2.42 (s, 3 H),2.20 (s, 3 H); MS m/z598.2 (M + 1). |
| 13 | 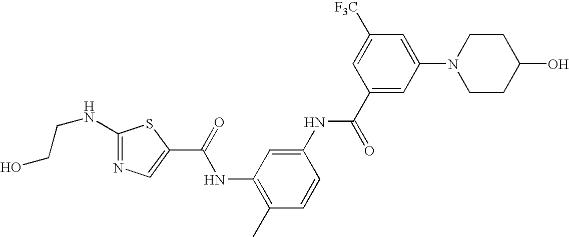 | ¹H NMR 400 MHz(MeOH-d₄) δ 8.19 (s, 1 H),8.01 (s, 1 H), 7.92 (s, 1 H),7.81 (s, 1 H), 7.73 (d, 1 H),7.58 (s, 1 H), 7.50 (d, 1 H),4.00 (m, 3 H), 3.76 (m,2 H), 3.28 (m, 4 H), 2.50 (s,3 H), 2.23 (m, 2 H), 1.89(m, 2 H); MS m/z 564.1(M + 1). |
| 14 | 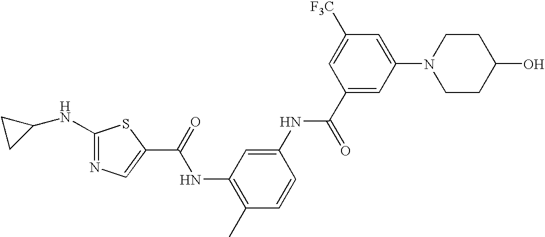 | ¹H NMR 400 MHz(DMSO-d₆) δ 9.60 (s, 1 H),8.59 (s, 1 H), 8.00 (s, 1 H),7.76 (s, 1 H), 7.69 (s, 1 H),7.58 (d, 1 H), 7.56 (s, 1 H),7.32 (s, 1 H), 7.22 (d, 1 H),3.68 (m, 3 H), 3.03 (m,2 H), 2.57 (m, 1 H), 2.21 (s,3 H), 1.85 (m, 2 H), 1.47(m, 2 H), 0.88 (m, 2 H),0.56 (m, 2 H); MS m/z560.4 (M + 1). |
| 15 | 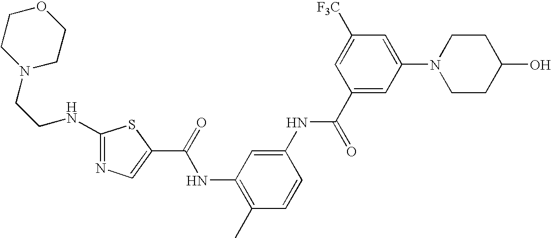 | ¹H NMR 400 MHz(MeOH-d₄) δ 7.90 (s, 1 H),7.76 (s, 1 H), 7.71 (s, 1 H),7.59 (s, 1 H), 7.48 (d, 1 H),7.33 (s, 1 H), 7.23 (d, 1 H),3.90 (br, 3 H), 3.82 (m,4 H), 3.73 (m, 2 H), 3.41(m, 4 H), 3.09 (m, 2 H),2.22 (s, 3 H), 1.97 (m, 2 H),1.43 (m, 2 H); MS m/z633.3 (M + 1). |
| 16 | 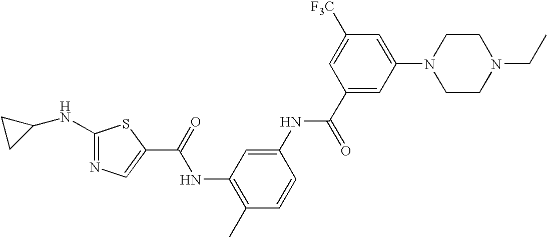 | ¹H NMR 400 MHz(DMSO-d₆) δ 9.68 (br,1 H), 9.59 (s, 1 H), 8.60 (s,1 H), 8.00 (s, 1 H), 7.78 (s,1 H), 7.75 (s, 1 H), 7.71 (s,1 H), 7.59 (d, 1 H), 7.50 (s,1 H), 7.22 (d, 1 H), 4.11 (m,2 H), 3.60 (br, 2 H), 3.24(br, 4 H), 2.55 (br, 1 H),2.20 (s, 3 H), 1.24 (t, 3 H),0.78 (m, 2 H), 0.59 (m,2 H); MS m/z573.3 (M + 1). |
| 17 | 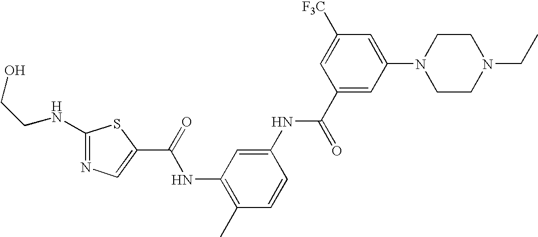 | ¹H NMR 400 MHz(MeOH-d₄) δ 7.87 (s, 1 H),7.77 (s, 1 H), 7.75 (s, 1 H),7.71 (s, 1 H), 7.51 (d, 1 H),7.46 (s, 1 H), 7.24 (d, 1 H),4.50 (br, 2 H), 3.72 (m,2 H), 3.68 (br, 2 H), 3.45(m, 2 H), 3.22 (br, 6 H),2.23 (s, 3 H), 1.38 (t, 3 H);MS m/z 577.5 (M + 1). |
| 18 | 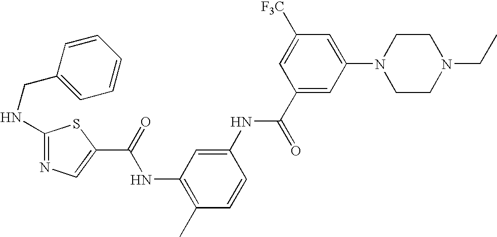 | ¹H NMR 400 MHz(DMSO-d₆) δ 9.66 (br,1 H), 9.58 (s, 1 H), 8.76 (t,1 H), 7.93 (s, 1 H), 7.72 (m,3 H), 7.57 (d, 1 H), 7.50 (s,1 H), 7.34 (m, 4 H), 7.23(m, 2 H), 4.50 (d, 2 H), 4.12(br, 2 H), 3.61 (br, 2 H),3.21 (br, 2 H), 3.13 (br,4 H), 2.17 (s, 3 H), 1.24 (t,3 H); MS m/z 623.3 (M + 1). |
| 19 | 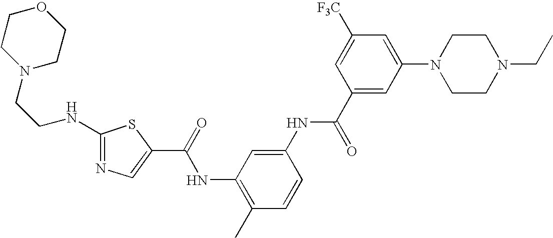 | ¹H NMR 400 MHz(DMSO-d₆) δ 9.80 (br,1 H), 9.69 (s, 1 H), 8.40 (t,1 H), 7.99 (s, 1 H), 7.78 (s,1 H), 7.70 (s, 1 H), 7.55 (d,1 H), 7.51 (s, 1 H), 7.26 (d,1 H), 4.11 (br, 2 H), 3.82(br, 4 H), 3.70 (br, 2 H),3.60 (br, 2 H), 3.35 (br,4 H), 3.23 (br, 2 H), 3.15(br, 6 H), 2.20 (s, 3 H), 1.25(t, 3 H); MS m/z 646.3(M + 1). |
| 20 | 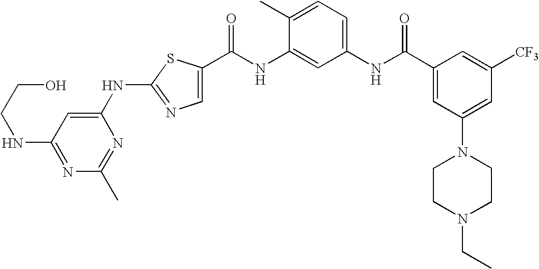 | ¹H NMR 400 MHz(MeOH-d₄) δ 8.17 (s, 1 H),7.80 (s, 1 H), 7.79 (s, 1 H),7.76 (s, 1 H), 7.54 (d, 1 H),7.48 (s, 1 H), 7.29 (d, 1 H),6.29 (br, 1 H), 4.07 (br,2 H), 3.76 (m, 2 H), 3.70(br, 2 H), 3.50 (br, 2 H),3.28 (br, 6 H), 2.60 (s, 3 H),2.30 (s, 3 H), 1.40 (t, 3 H);MS m/z 684.5 (M + 1). |
| 21 | 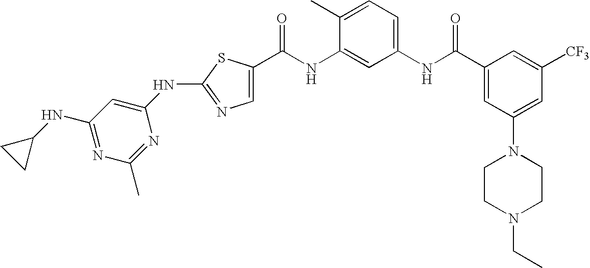 | ¹H NMR 400 MHz(MeOH-d₄) δ 8.19 (s, 1 H),7.81 (s, 1 H), 7.79 (s, 1 H),7.77 (s, 1 H), 7.54 (d, 1 H),7.48 (s, 1 H), 7.29 (d, 1 H),6.49 (br, 1 H), 4.08 (br,2 H), 3.70 (br, 2 H), 3.25(br, 6 H), 2.69 (br, 1 H),2.64 (s, 3 H), 2.30 (s, 3 H),1.40 (s, 3 H), 0.95 (br, 2 H),0.71 (br, 2 H); MS m/z680.5 (M + 1). |
| 22 | 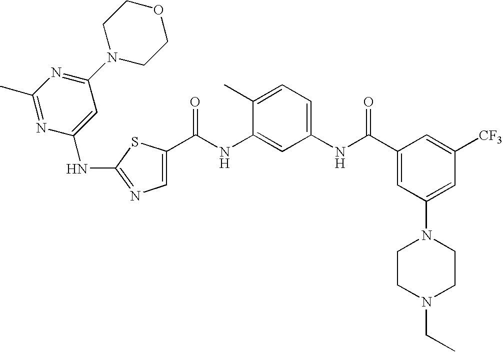 | ¹H NMR 400 MHz(MeOH-d₄) δ 8.15 (s, 1 H),7.81 (s, 1 H), 7.80 (s, 1 H),7.78 (s, 1 H), 7.54 (d, 1 H),7.49 (s, 1 H), 7.29 (d, 1 H),6.22 (br, 1 H), 4.07 (br,2 H), 3.79 (br, 4 H), 3.70(br, 2 H), 3.69 (br, 4 H),3.25 (br, 6 H), 2.57 (s, 3 H),2.30 (s, 3 H), 1.41 (t, 3 H);MS m/z 710.5 (M + 1). |
| 23 | 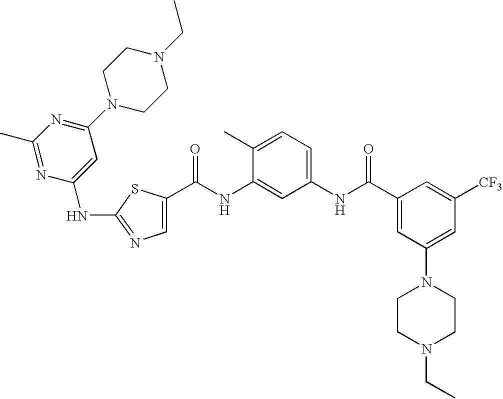 | ¹H NMR 400 MHz(MeOH-d₄) δ 8.13 (s, 1 H),7.83 (s, 1 H), 7.81 (s, 1 H),7.76 (s, 1 H), 7.53 (d, 1 H),7.47 (s, 1 H), 7.30 (d, 1 H),6.20 (s, 1 H), 4.60 (br, 2 H),4.07 (br, 2 H), 3.70 (br,2 H), 3.60 (br, 2 H), 3.29(br, 12 H), 2.52 (s, 3 H),2.30 (s, 3 H), 1.40 (m, 6 H);MS m/z 737.6 (M + 1). |
| 24 | 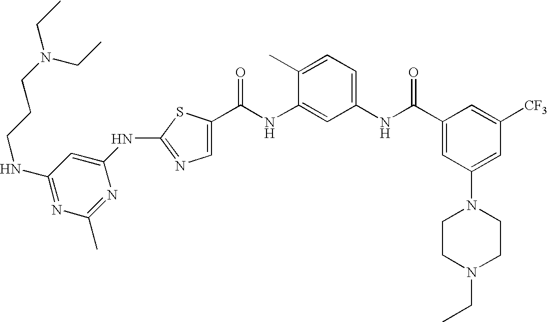 | ¹H NMR 400 MHz(MeOH-d₄) δ 8.17 (s, 1 H),7.82 (s, 1 H), 7.80 (s, 1 H),7.77 (s, 1 H), 7.53 (d, 1 H),7.48 (s, 1 H), 7.30 (d, 1 H),6.19 (s, 1 H), 4.08 (br, 2 H),3.71 (br, 2 H), 3.49 (br,2 H), 3.27 (br, 12 H), 2.60(s, 3 H), 2.30 (s, 3 H), 2.09(br, 2 H), 1.41 (t, 3 H), 1.32(t, 6 H); MS m/z 753.6 (M + 1). |
| 25 | 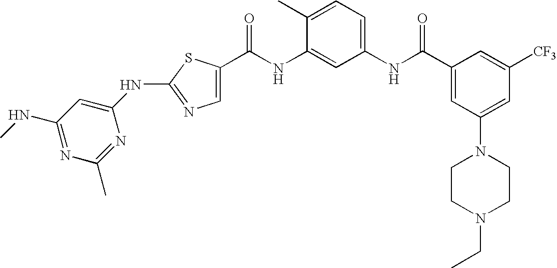 | ¹H NMR 400 MHz(MeOH-d₆) δ 8.15 (s, 1 H),7.79 (s, 1 H), 7.78 (s, 1 H),7.76 (s, 1 H), 7.54 (d, 1 H),7.48 (s, 1 H), 7.30 (d, 1 H),6.13 (br, 1 H), 4.08 (br,2 H), 3.71 (br, 2 H), 3.30(br, 6 H), 2.98 (s, 3 H), 2.61(s, 3 H), 2.30 (s, 3 H), 1.40(s, 3 H); MS m/z 654.5 (M + 1). |
| 26 | 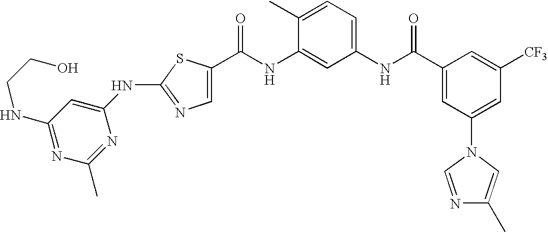 | ¹H NMR 400 MHz(DMSO-d₆) δ 9.70 (s, 1 H),9.40 (s, 1 H), 8.46 (s, 1 H),8.30 (s, 1 H), 8.29 (s, 1 H),8.11 (s, 1 H), 8.00 (s, 1 H),7.70 (s, 1 H), 7.50 (br, 1 H),7.49 (d, 1 H), 7.18 (d, 1 H),5.93 (br, 1 H), 3.39 (br,2 H), 3.20 (br, 2 H), 2.30 (s,3 H), 2.20 (s, 3 H), 2.11 (s,3 H); MS m/z 652.5 (M + 1). |
| 27 | 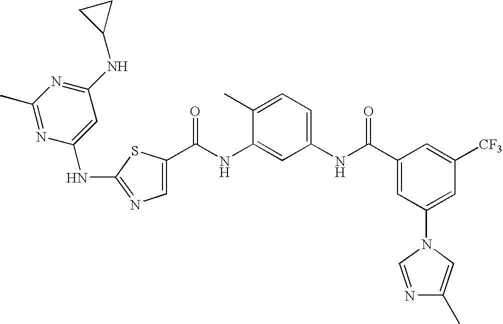 | ¹H NMR 400 MHz(DMSO-d₆) δ 9.78 (s, 1 H),9.40 (s, 1 H), 8.51 (s, 1 H),8.35 (s, 1 H), 8.34 (s, 1 H),8.17 (s, 1 H), 8.08 (s, 1 H),7.88 (br, 1 H), 7.77 (s, 1 H),7.54 (d, 1 H), 7.22 (d, 1 H),6.12 (s, 1 H), 2.45 (br, 1 H),2.36 (s, 3 H), 2.28 (s, 3 H),2.19 (s, 3 H), 0.71 (br, 2 H),0.43 (br, 2 H); MS m/z648.2 (M + 1). |
| 28 | 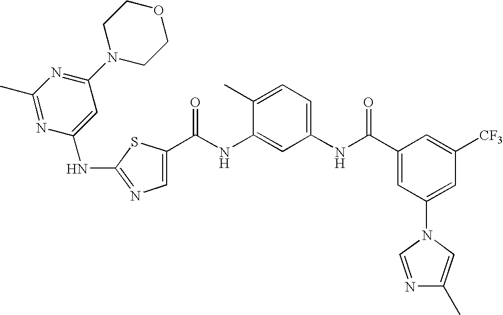 | ¹H NMR 400 MHz(DMSO-d₆) δ 9.80 (s, 1 H),9.67 (s, 1 H), 8.61 (s, 1 H),8.46 (s, 1 H), 8.44 (s, 1 H),8.23 (s, 1 H), 8.18 (s, 1 H),7.82 (s, 1 H), 7.61 (d, 1 H),7.30 (d, 1 H), 6.09 (s, 1 H),3.69 (m, 4 H), 3.50 (m,4 H), 2.43 (s, 3 H), 2.37 (s,3 H), 2.25 (s, 3 H); MS m/z678.2 (M + 1). |
| 29 | 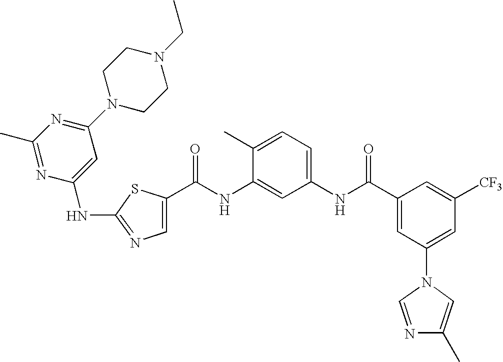 | ¹H NMR 400 MHz(DMSO-d₆) δ 9.85 (br,1 H), 9.80 (s, 1 H), 9.59 (s,1 H), 8.60 (s, 1 H), 8.44 (s,1 H), 8.43 (s, 1 H), 8.23 (s,1 H), 8.18 (s, 1 H), 7.85 (s,1 H), 7.60 (d, 1 H), 7.30 (d,1 H), 6.19 (s, 1 H), 4.39 (br,2 H), 3.60 (br, 2 H), 3.21(br, 4 H), 3.02 (br, 2 H),2.43 (s, 3 H), 2.36 (s, 3 H),2.25 (s, 3 H), 1.23 (t, 3 H);MS m/z 705.3 (M + 1). |
| 30 | 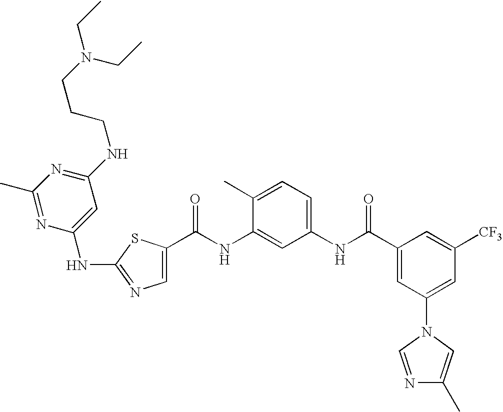 | ¹H NMR 400 MHz(DMSO-d₆) δ 9.80 (s, 1 H),9.58 (s, 1 H), 9.20 (br, 1 H),8.60 (s, 1 H), 8.42 (s, 1 H),8.41 (s, 1 H), 8.22 (s, 1 H),8.15 (s, 1 H), 7.86 (s, 1 H),7.61 (d, 1 H), 7.52 (br, 1 H),7.30 (d, 1 H), 5.96 (s, 1 H),3.32 (br, 2 H), 3.12 (br,6 H), 2.41 (s, 3 H), 2.37 (s,3 H), 2.27 (s, 3 H), 1.89 (br,2 H), 1.20 (t, 6 H); MS m/z721.5 (M + 1). |
| 31 | 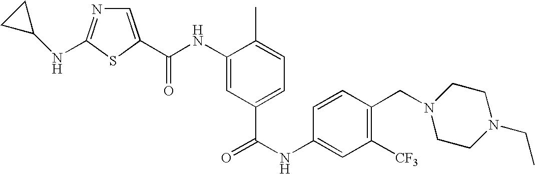 | MS m/z 587.3 (M + 1) |
| 32 | 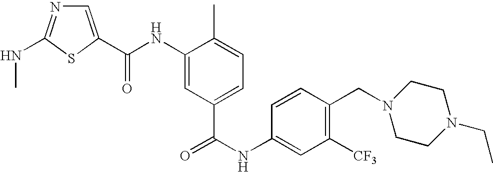 | MS m/z 561.3 (M + 1) |
| 33 | 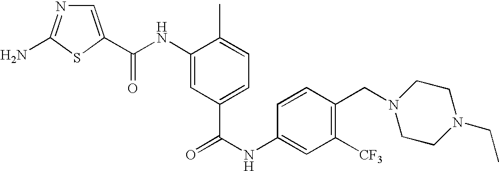 | MS m/z 547.3 (M + 1) |
| 34 | 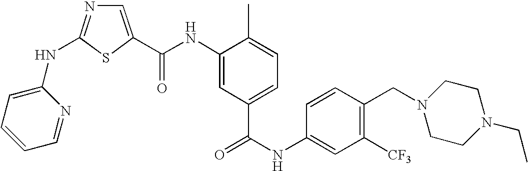 | MS m/z 624.3 (M + 1) |
| 35 | 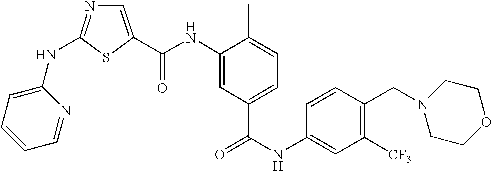 | MS m/z 597.2 (M + 1) |
| 36 | 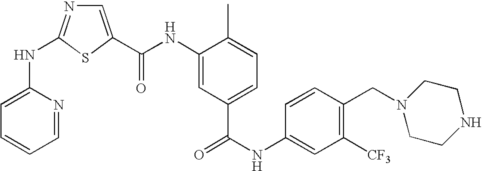 | MS m/z 596.2 (M + 1) |
| 37 | 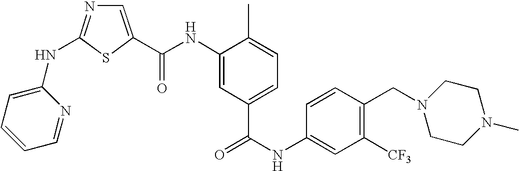 | MS m/z 610.2 (M + 1) |
| 38 | 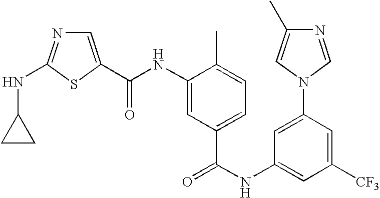 | MS m/z 541.2 (M + 1) |
| 39 | 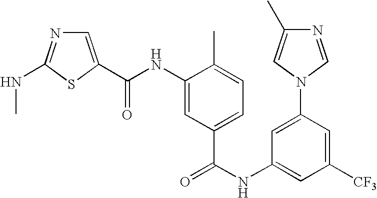 | MS m/z 515.4 (M + 1) |
| 40 | 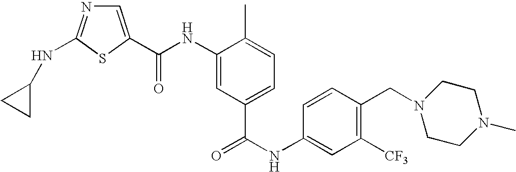 | MS m/z 573.2 (M + 1) |
| 41 | 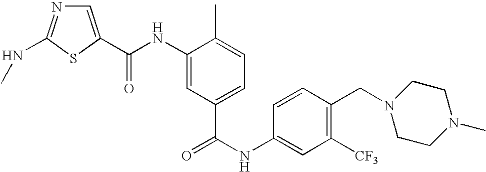 | MS m/z 547.2 (M + 1) |
| 42 | 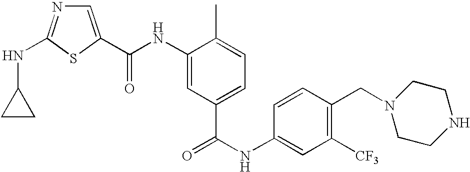 | MS m/z 559.2 (M + 1) |
| 43 | 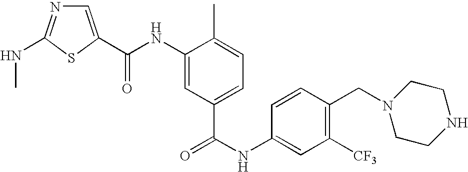 | MS m/z 533.2 (M + 1) |
| 44 | 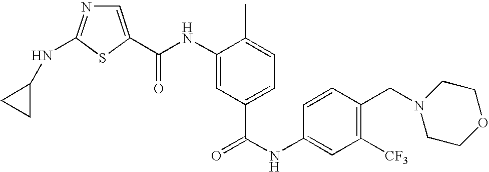 | MS m/z 560.2 (M + 1) |
| 45 | 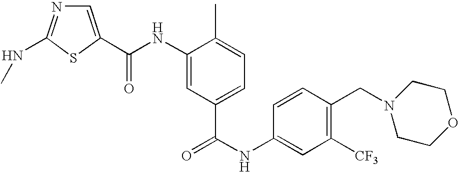 | MS m/z 534.2 (M + 1) |
| 46 | 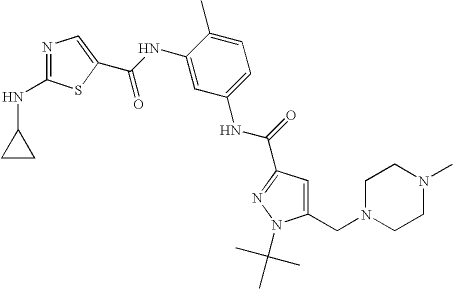 | MS m/z 551.3 (M + 1) |
| 47 | 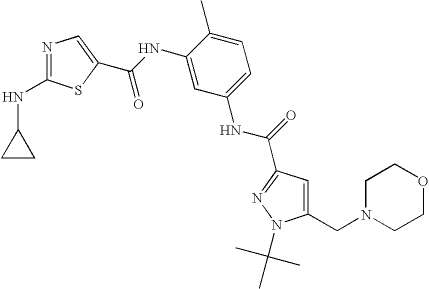 | MS m/z 538.3 (M + 1) |
| 48 | 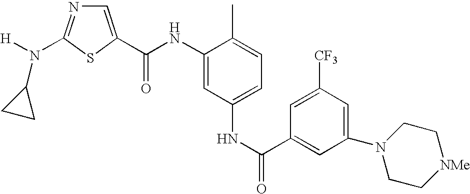 | MS m/z 559.2 (M + 1) |
| 49 | 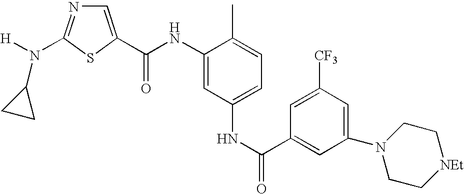 | MS m/z 573.2 (M + 1) |
| 50 | 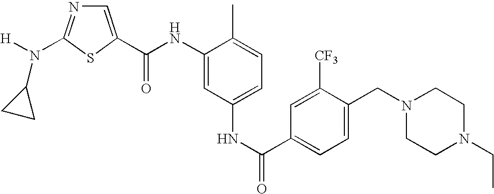 | MS m/z 587.2 (M + 1) |
| 51 | 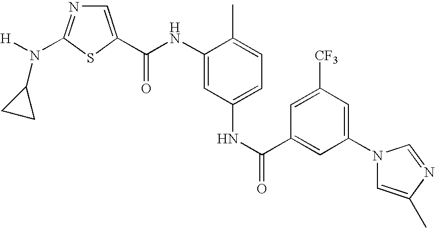 | MS m/z 541.2 (M + 1) |
| 52 | 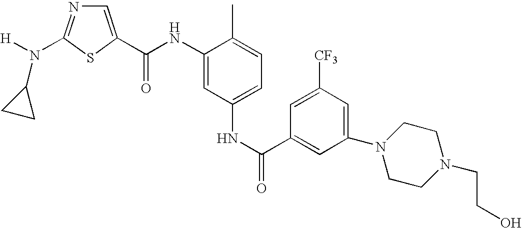 | MS m/z 589.2 (M + 1) |
| 53 | 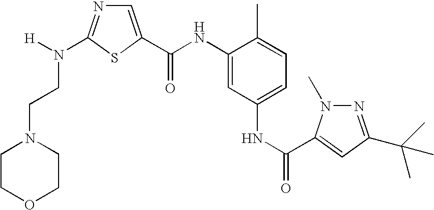 | MS m/z 526.3 (M + 1) |
| 54 | 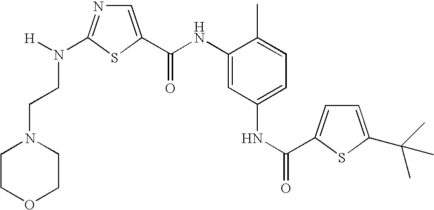 | MS m/z 568.1 (M + 1) |
| 55 | 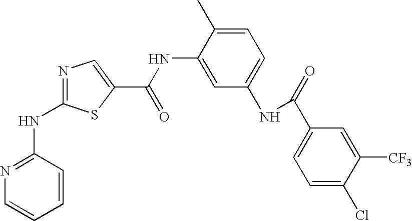 | MS m/z 532.1 (M + 1) |
| 56 | 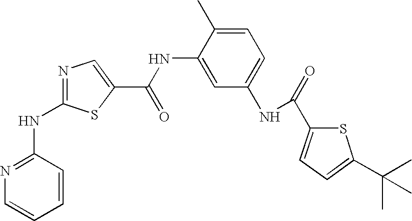 | MS m/z 492.1 (M + 1) |
| 57 | 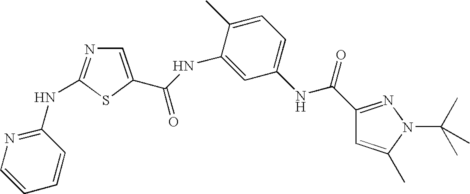 | MS m/z 490.2 (M + 1) |
| 58 | 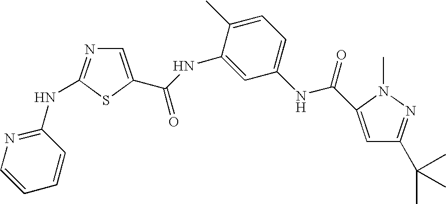 | MS m/z 490.2 (M + 1) |
| 59 | 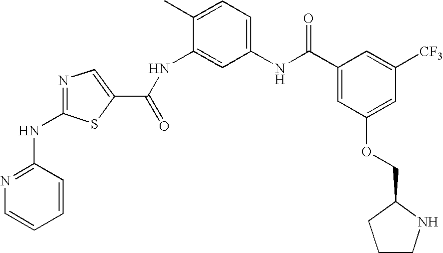 | MS m/z 697.2 (M + 1) |
| 60 | 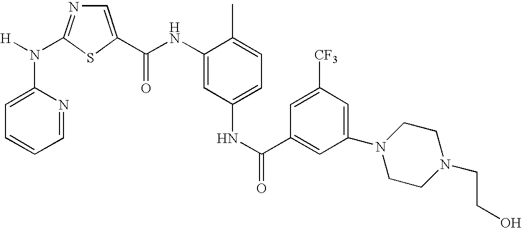 | MS m/z 626.2 (M + 1) |
| 61 | 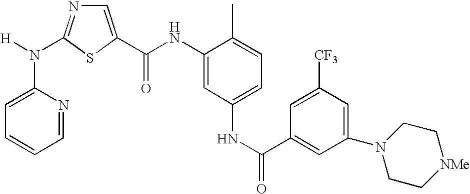 | MS m/z 592.2 (M + 1) |
| 62 | 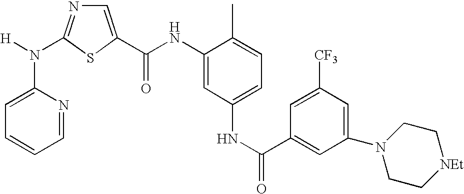 | MS m/z 610.2 (M + 1) |
| 63 | 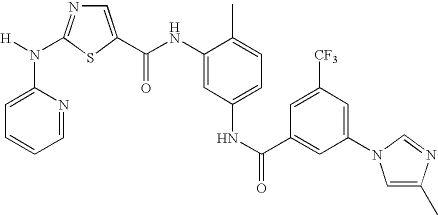 | MS m/z 578.2 (M + 1) |
| 64 | 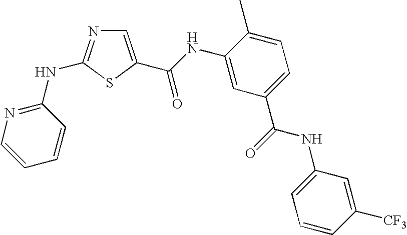 | MS m/z 498.1 (M + 1) |
| 65 | 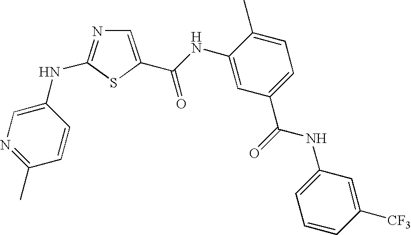 | MS m/z 512.1 (M + 1) |
| 66 | 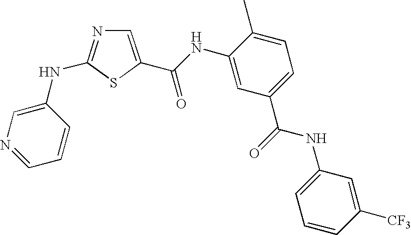 | MS m/z 498.1 (M + 1) |
| 67 | 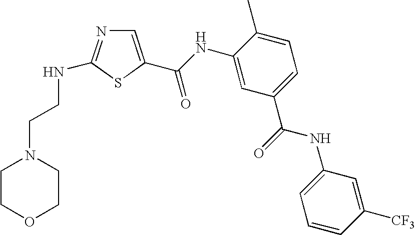 | MS m/z 534.2 (M + 1) |
| 68 | 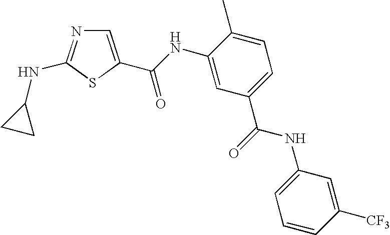 | MS m/z 461.1 (M + 1) |
| 69 | 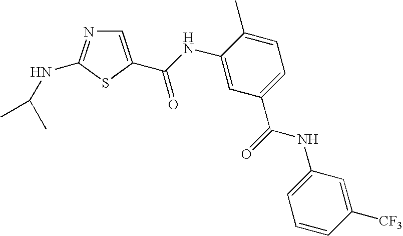 | MS m/z 463.1 (M + 1) |
| 70 | 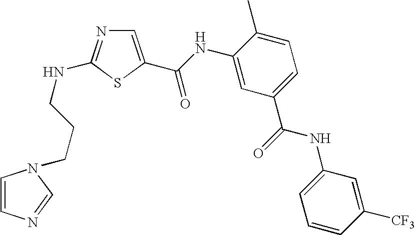 | MS m/z 529.2 (M + 1) |
| 71 | 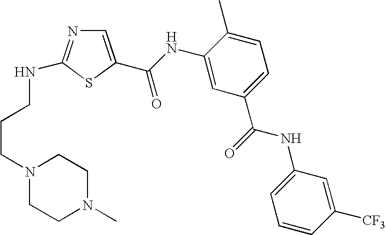 | MS m/z 561.2 (M + 1) |
| 72 | 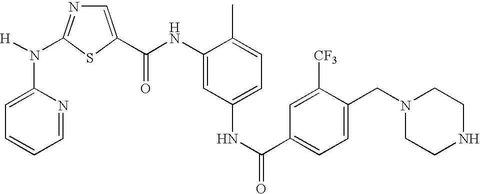 | MS m/z 596.2 (M + 1) |
| 73 | 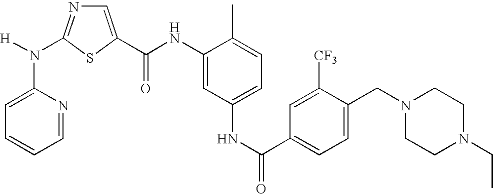 | MS m/z 624.2 (M + 1) |
| 74 | 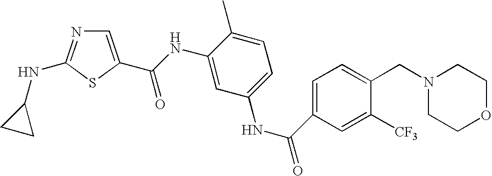 | MS m/z 560.2 (M + 1) |
| 75 | 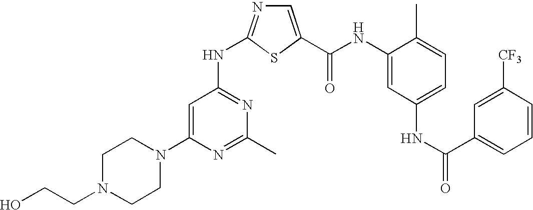 | MS m/z 641.2 (M + 1) |
| 76 | 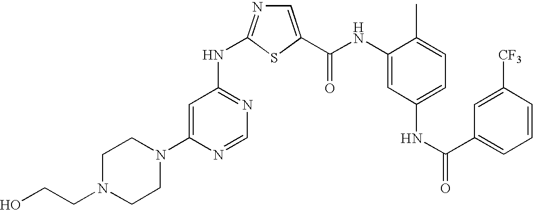 | MS m/z 627.2 (M + 1) |
| 77 | 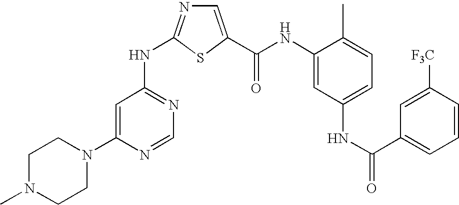 | MS m/z 597.2 (M + 1) |
| 78 | 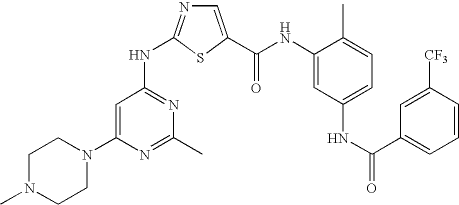 | MS m/z 611.2 (M + 1) |
| 79 | 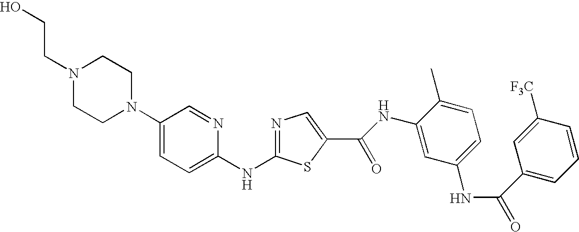 | MS m/z 626.2 (M + 1) |
| 80 | 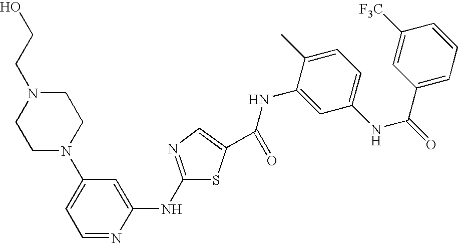 | MS m/z 626.2 (M + 1) |
| 81 | 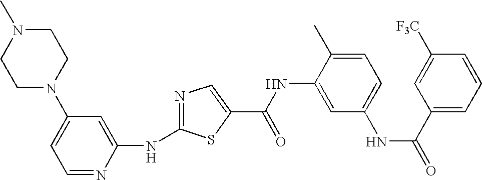 | MS m/z 596.2 (M + 1) |
| 82 | 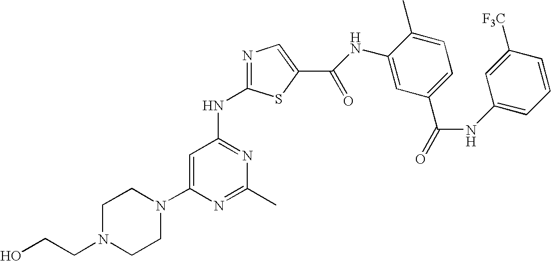 | MS m/z 641.2 (M + 1) |
| 83 | 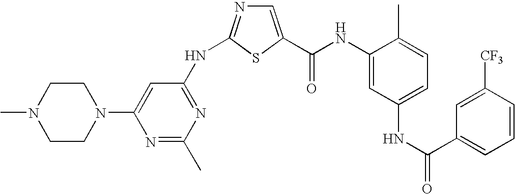 | MS m/z 611.2 (M + 1) |
| 84 | 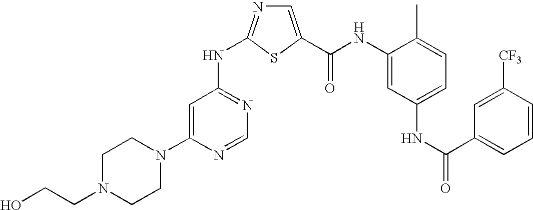 | MS m/z 627.2 (M + 1) |
| 85 | 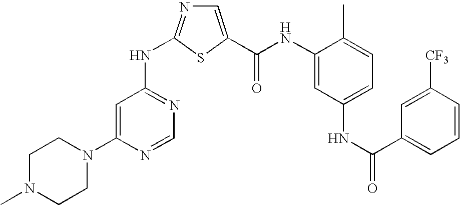 | MS m/z 597.2 (M + 1) |
| 86 | 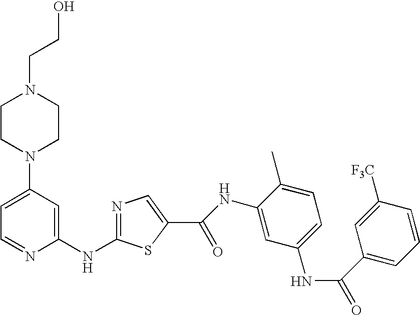 | MS m/z 626.2 (M + 1) |
| 87 | 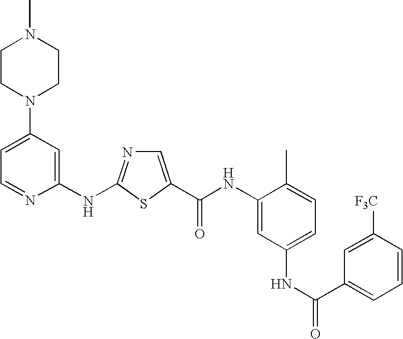 | MS m/z 596.2 (M + 1) |
| 88 | 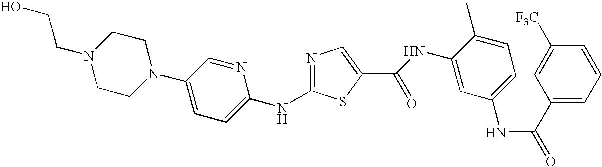 | MS m/z 626.2 (M + 1) |
| 89 | 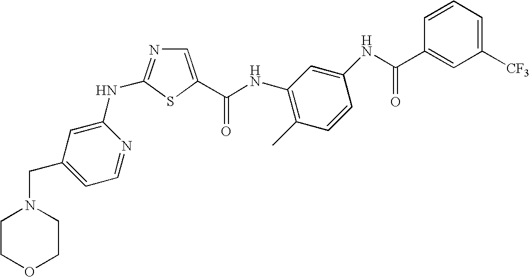 | MS m/z 597.2 (M + 1) |
| 90 | 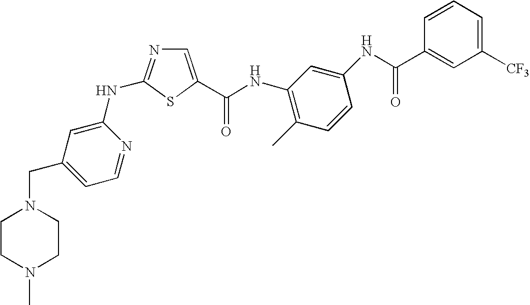 | MS m/z 610.3 (M + 1) |
| 91 | 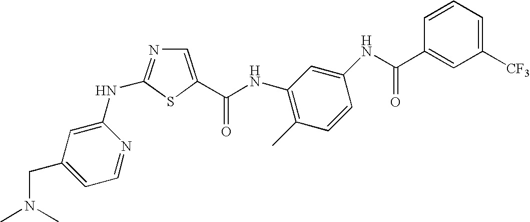 | MS m/z 555.2 (M + 1) |
| 92 | 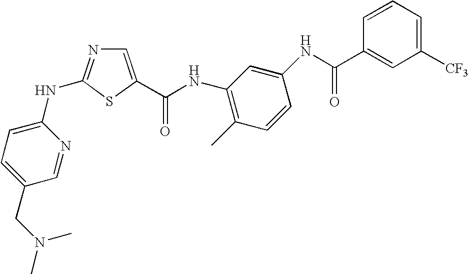 | MS m/z 555.2 (M + 1) |
| 93 | 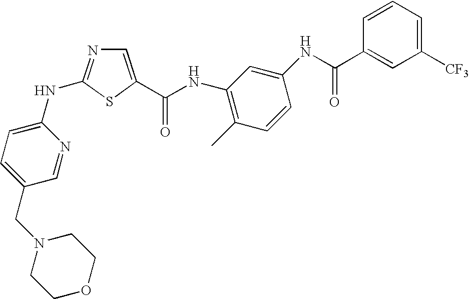 | MS m/z 597.2 (M + 1) |
| 94 | 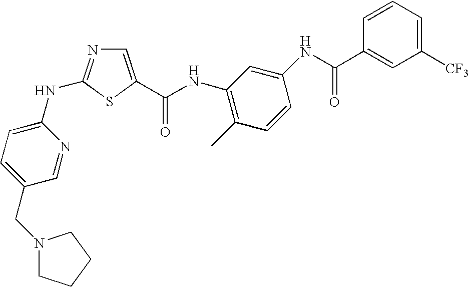 | MS m/z 581.2 (M + 1) |

### Assays

Compounds of the present invention are assayed to measure their capacity to selectively inhibit cell proliferation of Ba/F3 cells expressing BCR-Abl (Ba/F3-p210) compared with parental BaF3 cells. Compounds selectively inhibiting the proliferation of these BCR-Abl transformed cells are tested for anti-proliferative activity on Ba/F3 cells expressing either wild type or the mutant forms of Bcr-abl found in Gleevec resistant patients (mutations G250E, E255V, T315I, F317L and M351T).

In addition, compounds are assayed to measure their capacity to inhibit Abl, Bcr-Abl, FGFR3, PDGFRβ, Flt3 and b-Raf kinases.

### Inhibition of Cellular BCR-Abl Dependent Proliferation (High Throughput Method)

The murine cell line used is the Ba/F3 murine pro-B cell line transformed with BCR-Abl cDNA (Ba/F3-p210). These cells are maintained in RPMI/10% fetal calf serum (RPMI/FCS) supplemented with penicillin 50 μg/mL, streptomycin 50 μg/mL and L-glutamine 200 mM. Untransformed Ba/F3 Ba/F3 cells are similarly maintained with the addition of murine recombinant IL3.

### Inhibition of Cellular BCR-Abl Dependent Proliferation

Ba/F3-p210 cells are plated into 96 well TC plates at a density of 15,000 cells per well. 50 μL of two fold serial dilutions of the test compound (Cₘₐₓ, is −10 μM) are added to each well (STI571 is included as a positive control). After incubating the cells for 48 hours at 37° C., 5% CO₂, 15 μL of MTT (Promega) is added to each well and the cells are incubated for an additional 5 hours. The optical density at 570 nm is quantified spectrophotometrically and IC₅₀ values, the concentration of compound required for 50% inhibition, determined from a dose response curve.

### Effect on Cell Cycle Distribution

Ba/F3 and Ba/F3-p210 cells are plated into 6 well TC plates at 2.5×10⁶ cells per well in 5 ml of medium and test compound at 1 or 10 μM is added (STI571 is included as a control). The cells are then incubated for 24 or 48 hours at 37° C., 5% CO₂. 2 ml of cell suspension is washed with PBS, fixed in 70% EtOH for 1 hour and treated with PBS/EDTA/RNase A for 30 minutes. Propidium iodide (Cf=10 μg/ml) is added and the fluorescence intensity is quantified by flow cytometry on the FACScalibur system (BD Biosciences). Test compounds of the present invention demonstrate an apoptotic effect on the Ba/F3-p210 cells but do not induce apoptosis in the Ba/F3 parental cells.

### Effect on Cellular BCR-Abl Autophosphorylation

BCR-Abl autophosphorylation is quantified with capture Elisa using a c-abl specific capture antibody and an antiphosphotyrosine antibody. Ba/F3-p210 cells are plated in 96 well TC plates at 2×10⁵ cells per well in 50 μL of medium. 50 μL of two fold serial dilutions of test compounds (Cₘₐₓ is 10 μM) are added to each well (STI571 is included as a positive control). The cells are incubated for 90 minutes at 37° C., 5% CO₂. The cells are then treated for 1 hour on ice with 150 μL of lysis buffer (50 mM Tris-HCl, pH 7.4, 150 mM NaCl, 5 mM EDTA, 1 mM EGTA and 1% NP-40) containing protease and phosphatase inhibitors. 50 μL of cell lysate is added to 96 well optiplates previously coated with anti-abl specific antibody and blocked. The plates are incubated for 4 hours at 4° C. After washing with TBS-Tween 20 buffer, 50 μL of alkaline-phosphatase conjugated anti-phosphotyrosine antibody is added and the plate is further incubated overnight at 4° C. After washing with TBS-Tween 20 buffer, 90 μL of a luminescent substrate are added and the luminescence is quantified using the Acquest™ system (Molecular Devices). Test compounds of the invention that inhibit the proliferation of the BCR-Abl expressing cells, inhibit the cellular BCR-Abl autophosphorylation in a dose-dependent manner.

### Effect on Proliferation of Cells Expressing Mutant Forms of Bcr-abl

Compounds of the invention are tested for their antiproliferative effect on Ba/F3 cells expressing either wild type or the mutant forms of BCR-Abl (G250E, E255V, T315I, F317L, M351T) that confers resistance or diminished sensitivity to STI571. The antiproliferative effect of these compounds on the mutant-BCR-Abl expressing cells and on the non transformed cells were tested as described above. The IC₅₀ values of the compounds lacking toxicity on the untransformed cells were determined from the dose response curves obtained as describe above.

### FGFR3 (Enzymatic Assay)

Kinase activity assay with purified FGFR3 (Upstate) is carried out in a final volume of 10 μL containing 0.25 μg/mL of enzyme in kinase buffer (30 mM Tris-HCl pH7.5, 15 mM MgCl₂, 4.5 mM MnCl₂, 15 μM Na₃VO₄ and 50 μg/mL BSA), and substrates (5 μg/mL biotin-poly-EY(Glu, Tyr) (CIS-US, Inc.) and 3 μM ATP). Two solutions are made: the first solution of 5 μl contains the FGFR3 enzyme in kinase buffer was first dispensed into 384-format ProxiPlate® (Perkin-Elmer) followed by adding 50 μL of compounds dissolved in DMSO, then 5 μg of second solution contains the substrate (poly-EY) and ATP in kinase buffer was added to each wells. The reactions are incubated at room temperature for one hour, stopped by adding 10 μL of HTRF detection mixture, which contains 30 mM Tris-HCl pH7.5, 0.5 M KF, 50 mM ETDA, 0.2 mg/mL BSA, 15 μg/mL streptavidin-XL665 (CIS-US, Inc.) and 150 ng/mL cryptate conjugated anti-phosphotyrosine antibody (CIS-US, Inc.). After one hour of room temperature incubation to allow for streptavidin-biotin interaction, time resolved florescent signals are read on Analyst GT (Molecular Devices Corp.). IC₅₀ values are calculated by linear regression analysis of the percentage inhibition of each compound at 12 concentrations (1:3 dilution from 50 μM to 0.28 nM). In this assay, compounds of the invention have an IC₅₀ in the range of 10 nM to 2 μM.

### FGFR3 (Cellular Assay)

Compounds of the invention are tested for their ability to inhibit transformed Ba/F3-TEL-FGFR3 cells proliferation, which is depended on FGFR3 cellular kinase activity. Ba/F3-TEL-FGFR3 are cultured up to 800,000 cells/mL in suspension, with RPMI 1640 supplemented with 10% fetal bovine serum as the culture medium. Cells are dispensed into 384-well format plate at 5000 cell/well in 50 μL culture medium. Compounds of the invention are dissolved and diluted in dimethylsufoxide (DMSO). Twelve points 1:3 serial dilutions are made into DMSO to create concentrations gradient ranging typically from 10 mM to 0.05 μM. Cells are added with 50 mL of diluted compounds and incubated for 48 hours in cell culture incubator. AlamarBlue® (TREK Diagnostic Systems), which can be used to monitor the reducing environment created by proliferating cells, are added to cells at final concentration of 10%. After additional four hours of incubation in a 37° C. cell culture incubator, fluorescence signals from reduced AlamarBlue® (Excitation at 530 nm, Emission at 580 nm) are quantified on Analyst GT (Molecular Devices Corp.). IC₅₀ values are calculated by linear regression analysis of the percentage inhibition of each compound at 12 concentrations.

### FLT3 and PDGFRβ(Cellular Assay)

The effects of compounds of the invention on the cellular activity of FLT3 and PDGFRβ are conducted using identical methods as described above for FGFR3 cellular activity, except that instead of using Ba/F3-TEL-FGFR3, Ba/F3-FLT3-ITD and Ba/F3-Tel-PDGFRβ are used, respectively.

b-Raf-Enzymatic Assay

Compounds of the invention are tested for their ability to inhibit the activity of b-Raf. The assay is carried out in 384-well MaxiSorp plates (NUNC) with black walls and clear bottom. The substrate, IκBα is diluted in DPBS (1:750) and 15 μl is added to each well. The plates are incubated at 4° C. overnight and washed 3 times with TBST (25 mM Tris, pH 8.0, 150 mM NaCl and 0.05% Tween-20) using the EMBLA plate washer. Plates are blocked by Superblock (15 μl/well) for 3 hours at room temperature, washed 3 times with TBST and pat-dried. Assay buffer containing 20 μM ATP (10 μl) is added to each well followed by 100 nl or 500 nl of compound. B-Raf is diluted in the assay buffer (1 μl into 25 μl) and 10 μl of diluted b-Raf is added to each well (0.4 μg/well). The plates are incubated at room temperature for 2.5 hours. The kinase reaction is stopped by washing the plates 6 times with TBST. Phosph-IκBα (Ser32/36) antibody is diluted in Superblock (1:10,000) and 15 μl is added to each well. The plates are incubated at 4° C. overnight and washed 6 times with TBST. AP-conjugated goat-anti-mouse IgG is diluted in Superblock (1:1,500) and 15 μl is added to each well. Plates are incubated at room temperature for 1 hour and washed 6 times with TBST. 154 of fluorescent Attophos AP substrate (Promega) is added to each well and plates are incubated at room temperature for 15 minutes. Plates are read on Acquest or Analyst GT using a Fluorescence Intensity Program (Excitation 455 nm, Emission 580 nm).

b-Raf-Cellular Assay

Compounds of the invention are tested in A375 cells for their ability to inhibit phosphorylation of MEK. A375 cell line (ATCC) is derived from a human melanoma patient and it has a V599E mutation on the B-Raf gene. The levels of phosphorylated MEK are elevated due to the mutation of B-Raf. Sub-confluent to confluent A375 cells are incubated with compounds for 2 hours at 37° C. in serum free medium. Cells are then washed once with cold PBS and lysed with the lysis buffer containing 1% Triton X100. After centrifugation, the supernatants are subjected to SDS-PAGE, and then transferred to nitrocellulose membranes. The membranes are then subjected to western blotting with anti-phospho-MEK antibody (ser217/221) (Cell Signaling). The amount of phosphorylated MEK is monitored by the density of phospho-MEK bands on the nitrocellulose membranes.

### Upstate KinaseProfiler™—Radio-Enzymatic Filter Binding Assay

Compounds of the invention are assessed for their ability to inhibit individual members of the kinase panel. The compounds are tested in duplicates at a final concentration of 10 μM following this generic protocol. Note that the kinase buffer composition and the substrates vary for the different kinases included in the “Upstate KinaseProfiler™” panel. Kinase buffer (2.5 μL, 10×-containing MnCl₂ when required), active kinase (0.001-0.01 Units; 2.5 μL), specific or Poly(Glu-4-Tyr) peptide (5-500 μM or 0.01 mg/ml) in kinase buffer and kinase buffer (50 μM; 5 μL) are mixed in an eppendorf on ice. A Mg/ATP mix (10 μL; 67.5 (or 33.75) mM MgCl₂, 450 (or 225) μM ATP and 1 μCi/μl [γ-³²P]-ATP (3000 Ci/mmol)) is added and the reaction is incubated at about 30° C. for about 10 minutes. The reaction mixture is spotted (20 μL) onto a 2 cm×2 cm P81 (phosphocellulose, for positively charged peptide substrates) or Whatman No. 1 (for Poly (Glu-4-Tyr) peptide substrate) paper square. The assay squares are washed 4 times, for 5 minutes each, with 0.75% phosphoric acid and washed once with acetone for 5 minutes. The assay squares are transferred to a scintillation vial, 5 ml scintillation cocktail are added and ³²P incorporation (cpm) to the peptide substrate is quantified with a Beckman scintillation counter. Percentage inhibition is calculated for each reaction.

Compounds of Formula I, in free form or in pharmaceutically acceptable salt form, exhibit valuable pharmacological properties, for example, as indicated by the in vitro tests described in this application. For example, compounds of Formula I preferably show an IC₅₀ in the range of 1×10⁻¹⁰ to 1×10⁻⁵ M, preferably less than 150 nM for at least one of the following kinases: Abl, Bcr-Abl, FGFR3, PDGFRβ, b-Raf, and Flt-3. For example:

(i) 2-[6-(4-ethyl-piperazin-1-yl)-2-methyl-pyrimidin-4-ylamino]-thiazole-5-carboxylic acid [2-methyl-5-(3-trifluoromethyl-benzoylamino)-phenyl]-amide (Example 8) has an IC₅₀ of 5 nM, 2.29 μM, 12 nM, 1.27 μM 5 nM and 5 nM for wild type, G250E, E255V, T315I, F317L and M351T Bcr-abl, respectively;

(ii) 2-[6-(4-ethyl-piperazin-1-yl)-2-methyl-pyrimidin-4-ylamino]-thiazole-5-carboxylic acid {2-methyl-5-[3-(4-methyl-imidazol-1-yl)-5-trifluoromethyl-benzoylamino]-phenyl}-amide (Example 29) has an IC₅₀ of 8 nM and 570 nM for wild type and T315I Bcr-Abl respectively;

(iii) 2-(2-methyl-6-morpholin-4-yl-pyrimidin-4-ylamino)-thiazole-5-carboxylic acid {2-methyl-5-[3-(4-methyl-imidazol-1-yl)-5-trifluoromethyl-benzoylamino]-phenyl}-amide (Example 28) has an IC₅₀ of 5 nM for PDGFRβ; and

(iv) 2-[6-(2-hydroxy-ethylamino)-2-methyl-pyrimidin-4-ylamino]-thiazole-5-carboxylic acid {2-methyl-5-[3-(4-methyl-imidazol-1-yl)-5-trifluoromethyl-benzoylamino]-phenyl}-amide (Compound 5) has an IC₅₀ of 41 nM for Flt-3.

Compounds of Formula I, at a concentration of 10 μM, preferably show a percentage inhibition of greater than 50%, preferably greater than about 70%, against one or more of the following kinases: Abl, Bcr-Abl, FGFR3, PDGFRβ, b-Raf, and Flt-3.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

We claim:

1. A compound of Formula I: 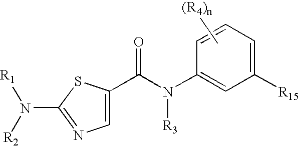
in which:
n is selected from 0, 1, 2, 3 and 4;
R₁ is selected from hydrogen, C₁₋₆alkyl, C₆₋₁₀aryl-C₀₋₄alkyl, C₅₋₁₀heteroaryl-C₀₋₄alkyl, C₃₋₁₂cycloalkyl-C₀₋₄-alkyl, C₃₋₈heterocycloalkyl-C₀₋₄alkyl and —XNR₇R₈;
wherein any aryl, heteroaryl, cycloalkyl or heterocycloalkyl of R₁ is optionally substituted with 1-3 radicals independently selected from halo, C₁₋₆alkyl, halo-substituted-C₁₋₆alkyl, C₁₋₆alkoxy, halo-substituted-C₁₋₆alkoxy, C₁₋₆alkylthio, halo-substituted-C₁₋₆ alkylthio, —XNR₇R₈, —XNR₇XNR₇R₈, —XNR₇R₉, C₆₋₁₀aryl-C₀₋₄-alkyl, C₅₋₁₀heteroaryl-C₀₋₄-alkyl, C₃₋₁₂cycloalkyl-C₀₋₄alkyl and C₃₋₈heterocycloalkyl-C₀₋₄alkyl; wherein any aryl, heteroaryl, cycloalkyl or heterocycloalkyl substituents on R₁ can be optionally substituted by 1 to 3 radicals independently selected from halo, C₁₋₆alkyl, halo-substituted-C₁₋₆alkyl, hydroxy-substituted-C₁₋₆alkyl, C₁₋₆alkoxy and halo-substituted-C₁₋₆alkoxy; and wherein any alkyl of R₁ can have a methylene replaced with O;
wherein each X is independently selected from a bond and C₁₋₆alkylene; R₇ and R₈ are independently selected from hydrogen and C₆alkyl; wherein any methylene of R₇ and R₈ can be replaced with O; wherein R₉ is selected from C₆₋₁₀aryl-C₀₋₄-alkyl, C₅₋₁₀heteroaryl-C₀₋₄alkyl, C₃₋₁₂cycloalkyl-C₀₋₄alkyl and C₃₋₈heterocycloalkyl-C₀₋₄alkyl;
R₂ is selected from hydrogen and C₁₋₆alkyl;
R₃ is selected from hydrogen and C₁₋₆alkyl;
R₄ is selected from halo, C₁₋₆alkyl, halo-substituted-C₁₋₆alkyl, C₁₋₆alkoxy, halo-substituted-C₁₋₆alkoxy, C₁₋₆alkylthio and halo-substituted-C₁₋₆alkylthio;
R₁₅ is selected from —NR₅Y(O)R₆ and —Y(O)NR₅R₆; wherein
Y is selected from C, S, S(O), P and P(O);
R₅ is selected from hydrogen and C₁₋₆alkyl; and
R₆ is selected from C₆₋₁₀aryl, C₅₋₁₀heteroaryl, C₃₋₁₂cycloalkyl and C₃₋₈heterocycloalkyl; wherein said aryl, heteroaryl, cycloalkyl or heterocycloalkyl of R₆ is optionally substituted with 1 to 3 substituents independently selected from halo, C₁₋₆alkyl, halo-substituted-C₁₋₆alkyl, C₁₋₆alkoxy, halo-substituted-C₁₋₆alkoxy, C₁₋₆alkylthio, halo-substituted-C₁₋₆alkylthio, C₆₋₁₀aryl-C₀₋₄alkyl, C₅₋₁₀heteroaryl-C₀₋₄alkyl, C₃₋₁₂cycloalkyl-C₀₋₄alkyl, C₃₋₈heterocycloalkyl-C₀₋₄alkoxy and C₃₋₈heterocycloalkyl-C₀₋₄alkyl; wherein the aryl, heteroaryl, cycloalkyl or heterocycloalkyl substituents on R₆ can be optionally be further substituted by 1 to 3 radicals independently selected from hydroxy, halo, C₁₋₆alkyl, halo-substituted-C₁₋₆alkyl, hydroxy-substituted-C₁₋₆alkyl, C₁₋₆alkoxy and halo-substituted-C₁₋₆alkoxy; and the pharmaceutically acceptable salts, hydrates, solvates, isomers and prodrugs thereof.

2. A compound of claim 1 of Formula Ia: 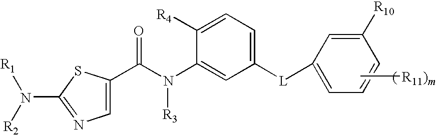
in which:
m is selected from 0 and 1;
R₁ is selected from hydrogen, C₁₋₆alkyl, C₆₋₁₀aryl-C₀₋₄alkyl, C₅₋₁₀ heteroaryl-C₀₋₄alkyl, C₃₋₁₂cycloalkyl-C₀₋₄alkyl, C₃₋₉heterocycloalkyl-C₀₋₄alkyl and —XNR₇R₉;
wherein any aryl, heteroaryl, cycloalkyl or heterocycloalkyl of R₁ is optionally substituted with 1 to 3 radicals independently selected from C₁₋₆alkyl, —XNR₇R₈, —XNR₇XNR₇R₈, —XNR₇R₉, C₅₋₁₀heteroaryl-C₀₋₄alkyl and C₃₋₈heterocycloalkyl-C₀₋₄alkyl; wherein any heteroaryl or heterocycloalkyl substituents on R₁ can be optionally substituted by 1 to 3 radicals independently selected from C₁₋₆alkyl and hydroxy-substituted-C₁₋₆alkyl;
and wherein any alkyl of R₁ can have a methylene replaced with O;
wherein each X is independently selected from a bond and C₁₋₆alkylene; R₇ and R₈ are independently selected from hydrogen and C₁₋₆alkyl; wherein any methylene of R₇ and R₈ can be replaced with O; wherein R₉ is C₃₋₁₂cycloalkyl-C₀₋₄alkyl;
R₂ is selected from hydrogen and C₁₋₆alkyl;
R₃ is selected from hydrogen and C₁₋₆alkyl;
R₄ is selected from halo, C₁₋₆alkyl, halo-substituted-C₁₋₆alkyl, C₁₋₆alkoxy and halo-substituted-C₁₋₆alkoxy;
L is selected from —NR₅C(O)— and —C(O)NR₅—;
R₅ is selected from hydrogen and C₁₋₆alkyl; and
R₁₀ is halo-substituted-C₁₋₆alkyl; and
R₁₁ is selected from hydrogen, halo, C₅₋₁₀heteroaryl and C₃₋₈heterocycloalkyl; wherein the heteroaryl or heterocycloalkyl substituents on R₁₀ can be optionally substituted by 1 to 3 radicals independently selected from hydroxy and C₁₋₆alkyl.

3. The compound of claim 2 in which R₁ is selected from hydrogen, methyl, isopropyl, imidazolyl-propyl, piperazinyl-propyl, pyridinyl, diethyl-amino-propyl, hydroxy-ethyl, pyrimidinyl, morpholino-propyl, phenyl, cyclopropyl, morpholino-ethyl, benzyl and morpholino; wherein any pyridinyl, imidazolyl, piperazinyl or pyrimidinyl of R₁ is optionally substituted with 1 to 3 radicals independently selected from methyl, methyl-amino, dimethyl-amino-methyl, cyclopropyl-amino, hydroxy-ethyl-amino, diethyl-amino-propyl-amino, pyrrolidinyl-methyl, morpholino, morpholino-methyl, piperazinyl methyl and piperazinyl; wherein any morpholino and piperazinyl substituent of R₁ is optionally further substituted by a radical selected from methyl, hydroxy-ethyl and ethyl; R₂, R₃ and R₅ are each hydrogen; and R₄ is methyl.

4. The compound of claim 3 in which m is selected from 0 and 1; R₁₀ is trifluoromethyl; and R₁₁ is selected from: halo; morpholino-methyl; piperazinyl optionally substituted with methyl, ethyl or hydroxyethyl; piperazinyl-methyl optionally substituted with methyl or ethyl; imidazolyl optionally substituted with methyl; pyrrolidinyl-methoxy; and piperidinyl optionally substituted with hydroxy.

5. The compound of claim 1 selected from: 2-(3-Diethylaminopropylamino)-thiazole-5-carboxylic acid [2-methyl-5-(3-trifluoromethyl-benzoylamino)-phenyl]-amide; 2-{6-[4-(2-Hydroxyethyl)-piperazin-1-yl]-2-methylpyrimidin-4-ylamino}-thiazole-5-carboxylic acid [2-methyl-5-(3-trifluoromethylbenzoylamino)-phenyl]-amide; 2-(2-Hydroxy-ethylamino)-thiazole-5-carboxylic acid {5-[3-(4-ethyl-piperazin-1-yl)-5-trifluoromethyl-benzoylamino]-2-methyl-phenyl}-amide; 2-{6-[4-(2-Hydroxy-ethyl)-piperazin-1-yl]-2-methyl-pyrimidin-4-ylamino}-thiazole-5-carboxylic acid [2-methyl-5-(3-trifluoromethyl-benzoylamino)-phenyl]-amide; 2-(3-Morpholin-4-yl-propylamino)-thiazole-5-carboxylic acid [2-methyl-5-(3-trifluoromethyl-benzoylamino)-phenyl]-amide; 2-(3-Diethylamino-propylamino)-thiazole-5-carboxylic acid [2-methyl-5-(3-trifluoromethyl-benzoylamino)-phenyl]-amide; 2-Phenylamino-thiazole-5-carboxylic acid [2-methyl-5-(3-trifluoromethyl-benzoylamino)-phenyl]-amide; 2-(2-Hydroxy-ethylamino)-thiazole-5-carboxylic acid {2-methyl-5-[3-(4-methyl-imidazol-1-yl)-5-trifluoromethyl-benzoylamino]-phenyl}-amide; 2-(3-Diethylamino-propylamino)-thiazole-5-carboxylic acid {2-methyl-5-[3-(4-methyl-imidazol-1-yl)-5-trifluoromethyl-benzoylamino]-phenyl}-amide; 2-(3-Morpholin-4-yl-propylamino)-thiazole-5-carboxylic acid {2-methyl-5-[3-(4-methyl-imidazol-1-yl)-5-trifluoromethyl-benzoylamino]-phenyl}-amide; 2-[6-(4-Ethyl-piperazin-1-yl)-2-methyl-pyrimidin-4-ylamino]-thiazole-5-carboxylic acid [2-methyl-5-(3-trifluoromethyl-benzoylamino)-phenyl]-amide; 2-(6-Cyclopropylamino-2-methyl-pyrimidin-4-ylamino)-thiazole-5-carboxylic acid [2-methyl-5-(3-trifluoromethyl-benzoylamino)-phenyl]-amide; 2-[6-(2-Hydroxy-ethylamino)-2-methyl-pyrimidin-4-ylamino]-thiazole-5-carboxylic acid [2-methyl-5-(3-trifluoromethyl-benzoylamino)-phenyl]-amide; 2-[6-(3-Diethylamino-propylamino)-2-methyl-pyrimidin-4-ylamino]-thiazole-5-carboxylic acid [2-methyl-5-(3-trifluoromethyl-benzoylamino)-phenyl]-amide; 2-(2-Methyl-6-morpholin-4-yl-pyrimidin-4-ylamino)-thiazole-5-carboxylic acid [2-methyl-5-(3-trifluoromethyl-benzoylamino)-phenyl]-amide; 2-(2-Hydroxy-ethylamino)-thiazole-5-carboxylic acid {5-[3-(4-hydroxy-piperidin-1-yl)-5-trifluoromethyl-benzoylamino]-2-methyl-phenyl}-amide; 2-Cyclopropylamino-thiazole-5-carboxylic acid {5-[3-(4-hydroxy-piperidin-1-yl)-5-trifluoromethyl-benzoylamino]-2-methyl-phenyl}-amide; 2-(2-Morpholin-4-yl-ethylamino)-thiazole-5-carboxylic acid {5-[3-(4-hydroxy-piperidin-1-yl)-5-trifluoromethyl-benzoylamino]-2-methyl-phenyl}-amide; 2-Cyclopropylamino-thiazole-5-carboxylic acid {5-[3-(4-ethyl-piperazin-1-yl)-5-trifluoromethyl-benzoylamino]-2-methyl-phenyl}-amide; 2-(2-Hydroxy-ethylamino)-thiazole-5-carboxylic acid {5-[3-(4-ethyl-piperazin-1-yl)-5-trifluoromethyl-benzoylamino]-2-methyl-phenyl}-amide; 2-Benzylamino-thiazole-5-carboxylic acid {5-[3-(4-ethyl-piperazin-1-yl)-5-trifluoromethyl-benzoylamino]-2-methyl-phenyl}-amide; 2-(2-Morpholin-4-yl-ethylamino)-thiazole-5-carboxylic acid {5-[3-(4-ethyl-piperazin-1-yl)-5-trifluoromethyl-benzoylamino]-2-methyl-phenyl}-amide; 2-[6-(2-Hydroxy-ethylamino)-2-methyl-pyrimidin-4-ylamino]-thiazole-5-carboxylic acid {5-[3-(4-ethyl-piperazin-1-yl)-5-trifluoromethyl-benzoylamino]-2-methyl-phenyl}-amide; 2-(6-Cyclopropylamino-2-methyl-pyrimidin-4-ylamino)-thiazole-5-carboxylic acid {5-[3-(4-ethyl-piperazin-1-yl)-5-trifluoromethyl-benzoylamino]-2-methyl-phenyl}-amide; 2-(2-Methyl-6-morpholin-4-yl-pyrimidin-4-ylamino)-thiazole-5-carboxylic acid {5-[3-(4-ethyl-piperazin-1-yl)-5-trifluoromethyl-benzoylamino]-2-methyl-phenyl}-amide; 2-[6-(4-Ethyl-piperazin-1-yl)-2-methyl-pyrimidin-4-ylamino]-thiazole-5-carboxylic acid {5-[3-(4-ethyl-piperazin-1-yl)-5-trifluoromethyl-benzoylamino]-2-methyl-phenyl}-amide; 2-[6-(3-Diethylamino-propylamino)-2-methyl-pyrimidin-4-ylamino]-thiazole-5-carboxylic acid {5-[3-(4-ethyl-piperazin-1-yl)-5-trifluoromethyl-benzoylamino]-2-methyl-phenyl}-amide; 2-(2-Methyl-6-methylamino-pyrimidin-4-ylamino)-thiazole-5-carboxylic acid {5-[3-(4-ethyl-piperazin-1-yl)-5-trifluoromethyl-benzoylamino]-2-methyl-phenyl}-amide; 2-[6-(2-Hydroxy-ethylamino)-2-methyl-pyrimidin-4-ylamino]-thiazole-5-carboxylic acid {2-methyl-5-[3-(4-methyl-imidazol-1-yl)-5-trifluoromethyl-benzoylamino]-phenyl}-amide; 2-(6-Cyclopropylamino-2-methyl-pyrimidin-4-ylamino)-thiazole-5-carboxylic acid {2-methyl-5-[3-(4-methyl-imidazol-1-yl)-5-trifluoromethyl-benzoylamino]-phenyl}-amide; 2-(2-Methyl-6-morpholin-4-yl-pyrimidin-4-ylamino)-thiazole-5-carboxylic acid {2-methyl-5-[3-(4-methyl-imidazol-1-yl)-5-trifluoromethyl-benzoylamino]-phenyl}-amide; 2-[6-(4-Ethyl-piperazin-1-yl)-2-methyl-pyrimidin-4-ylamino]-thiazole-5-carboxylic acid {2-methyl-5-[3-(4-methyl-imidazol-1-yl)-5-trifluoromethyl-benzoylamino]-phenyl}-amide; 2-[6-(3-Diethylamino-propylamino)-2-methyl-pyrimidin-4-ylamino]-thiazole-5-carboxylic acid {2-methyl-5-[3-(4-methyl-imidazol-1-yl)-5-trifluoromethyl-benzoylamino]-phenyl}-amide; 2-Cyclopropylamino-thiazole-5-carboxylic acid {5-[4-(4-ethyl-piperazin-1-ylmethyl)-3-trifluoromethyl-phenylcarbamoyl]-2-methyl-phenyl}-amide; 2-Methylamino-thiazole-5-carboxylic acid {5-[4-(4-ethyl-piperazin-1-ylmethyl)-3-trifluoromethyl-phenylcarbamoyl]-2-methyl-phenyl}-amide; 2-Amino-thiazole-5-carboxylic acid {5-[4-(4-ethyl-piperazin-1-ylmethyl)-3-trifluoromethyl-phenylcarbamoyl]-2-methyl-phenyl}-amide; 2-(Pyridin-2-ylamino)-thiazole-5-carboxylic acid {5-[4-(4-ethyl-piperazin-1-ylmethyl)-3-trifluoromethyl-phenylcarbamoyl]-2-methyl-phenyl}-amide; 2-(Pyridin-2-ylamino)-thiazole-5-carboxylic acid [2-methyl-5-(4-morpholin-4-ylmethyl-3-trifluoromethyl-phenylcarbamoyl)-phenyl]-amide; 2-(Pyridin-2-ylamino)-thiazole-5-carboxylic acid [2-methyl-5-(4-piperazin-1-ylmethyl-3-trifluoromethyl-phenylcarbamoyl)-phenyl]-amide; 2-(Pyridin-2-ylamino)-thiazole-5-carboxylic acid {2-methyl-5-[4-(4-methyl-piperazin-1-ylmethyl)-3-trifluoromethyl-phenylcarbamoyl]-phenyl}-amide; 2-Cyclopropylamino-thiazole-5-carboxylic acid {2-methyl-5-[3-(4-methyl-imidazol-1-yl)-5-trifluoromethyl-phenylcarbamoyl]-phenyl}-amide; 2-Methylamino-thiazole-5-carboxylic acid {2-methyl-5-[4-(4-methyl-piperazin-1-ylmethyl)-3-trifluoromethyl-phenylcarbamoyl]-phenyl}-amide; 2-Cyclopropylamino-thiazole-5-carboxylic acid [2-methyl-5-(4-piperazin-1-ylmethyl-3-trifluoromethyl-phenylcarbamoyl)-phenyl]-amide; 2-Methylamino-thiazole-5-carboxylic acid [2-methyl-5-(4-piperazin-1-ylmethyl-3-trifluoromethyl-phenylcarbamoyl)-phenyl]-amide; 2-Cyclopropylamino-thiazole-5-carboxylic acid [2-methyl-5-(4-morpholin-4-ylmethyl-3-trifluoromethyl-phenylcarbamoyl)-phenyl]-amide; 2-Methylamino-thiazole-5-carboxylic acid [2-methyl-5-(4-morpholin-4-ylmethyl-3-trifluoromethyl-phenylcarbamoyl)-phenyl]-amide; 2-Cyclopropylamino-thiazole-5-carboxylic acid (5-{[1-tert-butyl-5-(4-methyl-piperazin-1-ylmethyl)-1H-pyrazole-3-carbonyl]-amino}-2-methyl-phenyl)-amide; 2-Cyclopropylamino-thiazole-5-carboxylic acid {2-methyl-5-[3-(4-methyl-piperazin-1-yl)-5-trifluoromethyl-benzoylamino]-phenyl}-amide; 2-Methylamino-thiazole-5-carboxylic acid {2-methyl-5-[3-(4-methyl-imidazol-1-yl)-5-trifluoromethyl-phenylcarbamoyl]-phenyl}-amide; 2-Cyclopropylamino-thiazole-5-carboxylic acid {2-methyl-5-[4-(4-methyl-piperazin-1-ylmethyl)-3-trifluoromethyl-phenylcarbamoyl]-phenyl}-amide; 2-Cyclopropylamino-thiazole-5-carboxylic acid {5-[3-(4-ethyl-piperazin-1-yl)-5-trifluoromethyl-benzoylamino]-2-methyl-phenyl}-amide; 2-Cyclopropylamino-thiazole-5-carboxylic acid {5-[4-(4-ethyl-piperazin-1-ylmethyl)-3-trifluoromethyl-benzoylamino]-2-methyl-phenyl}-amide; 2-Cyclopropylamino-thiazole-5-carboxylic acid {2-methyl-5-[3-(4-methyl-imidazol-1-yl)-5-trifluoromethyl-benzoylamino]-phenyl}-amide; 2-Cyclopropylamino-thiazole-5-carboxylic acid (5-{3-[4-(2-hydroxy-ethyl)-piperazin-1-yl]-5-trifluoromethyl-benzoylamino}-2-methyl-phenyl)-amide; 2-(2-Morpholin-4-yl-ethylamino)-thiazole-5-carboxylic acid {5-[(5-tert-butyl-thiophene-2-carbonyl)-amino]-2-methyl-phenyl}-amide; 2-(Pyridin-2-ylamino)-thiazole-5-carboxylic acid {5-[(5-tert-butyl-thiophene-2-carbonyl)-amino]-2-methyl-phenyl}-amide; 2-(Pyridin-2-ylamino)-thiazole-5-carboxylic acid {5-[(5-tert-butyl-2-methyl-2H-pyrazole-3-carbonyl)-amino]-2-methyl-phenyl}-amide; 2-{5-[4-(2-Hydroxy-ethyl)-piperazin-1-yl]-pyridin-2-ylamino}-thiazole-5-carboxylic acid [2-methyl-5-(3-trifluoromethyl-benzoylamino)-phenyl]-amide; 2-(Pyridin-2-ylamino)-thiazole-5-carboxylic acid (5-{3-[4-(2-hydroxy-ethyl)-piperazin-1-yl]-5-trifluoromethyl-benzoylamino}-2-methyl-phenyl)-amide; 2-(Pyridin-2-ylamino)-thiazole-5-carboxylic acid {5-[3-(4-ethyl-piperazin-1-yl)-5-trifluoromethyl-benzoylamino]-2-methyl-phenyl}-amide; 2-(Pyridin-2-ylamino)-thiazole-5-carboxylic acid [2-methyl-5-(3-trifluoromethyl-phenylcarbamoyl)-phenyl]-amide; 2-(Pyridin-3-ylamino)-thiazole-5-carboxylic acid [2-methyl-5-(3-trifluoromethyl-phenylcarbamoyl)-phenyl]-amide; 2-Cyclopropylamino-thiazole-5-carboxylic acid [2-methyl-5-(3-trifluoromethyl-phenylcarbamoyl)-phenyl]-amide; 2-(3-Imidazol-1-yl-propylamino)-thiazole-5-carboxylic acid [2-methyl-5-(3-trifluoromethyl-phenylcarbamoyl)-phenyl]-amide; 2-(2-Morpholin-4-yl-ethylamino)-thiazole-5-carboxylic acid {5-[(5-tert-butyl-2-methyl-2H-pyrazole-3-carbonyl)-amino]-2-methyl-phenyl}-amide; 2-(Pyridin-2-ylamino)-thiazole-5-carboxylic acid [5-(4-chloro-3-trifluoromethyl-benzoylamino)-2-methyl-phenyl]-amide; 2-(Pyridin-2-ylamino)-thiazole-5-carboxylic acid {5-[(1-tert-butyl-5-methyl-1H-pyrazole-3-carbonyl)-amino]-2-methyl-phenyl}-amide; 2-(Pyridin-2-ylamino)-thiazole-5-carboxylic acid {2-methyl-5-[3-(pyrrolidin-2-ylmethoxy)-5-trifluoromethyl-benzoylamino]-phenyl}-amide; 2-(Pyridin-2-ylamino)-thiazole-5-carboxylic acid {2-methyl-5-[3-(4-methyl-piperazin-1-yl)-5-trifluoromethyl-benzoylamino]-phenyl}-amide; 2-(Pyridin-2-ylamino)-thiazole-5-carboxylic acid {2-methyl-5-[3-(4-methyl-imidazol-1-yl)-5-trifluoromethyl-benzoylamino]-phenyl}-amide; 2-(6-Methyl-pyridin-3-ylamino)-thiazole-5-carboxylic acid [2-methyl-5-(3-trifluoromethyl-phenylcarbamoyl)-phenyl]-amide; 2-(2-Morpholin-4-yl-ethylamino)-thiazole-5-carboxylic acid [2-methyl-5-(3-trifluoromethyl-phenylcarbamoyl)-phenyl]-amide; 2-Isopropylamino-thiazole-5-carboxylic acid [2-methyl-5-(3-trifluoromethyl-phenylcarbamoyl)-phenyl]-amide; 2-[3-(4-Methyl-piperazin-1-yl)-propylamino]-thiazole-5-carboxylic acid [2-methyl-5-(3-trifluoromethyl-phenylcarbamoyl)-phenyl]-amide; 2-(Pyridin-2-ylamino)-thiazole-5-carboxylic acid [2-methyl-5-(4-piperazin-1-ylmethyl-3-trifluoromethyl-benzoylamino)-phenyl]-amide; 2-(Pyridin-2-ylamino)-thiazole-5-carboxylic acid {5-[4-(4-ethyl-piperazin-1-ylmethyl)-3-trifluoromethyl-benzoylamino]-2-methyl-phenyl}-amide; 2-Cyclopropylamino-thiazole-5-carboxylic acid [2-methyl-5-(4-morpholin-4-ylmethyl-3-trifluoromethyl-benzoylamino)-phenyl]-amide; 2{6-[4-(2-Hydroxy-ethyl)-piperazin-1-yl]-2-methyl-pyrimidin-4-ylamino}-thiazole-5-carboxylic acid [2-methyl-5-(3-trifluoromethyl-benzoylamino)-phenyl]-amide; 2-[6-(4-Methyl-piperazin-1-yl)-pyrimidin-4-ylamino]-thiazole-5-carboxylic acid [2-methyl-5-(3-trifluoromethyl-benzoylamino)-phenyl]-amide; 2-{6-[4-(2-Hydroxy-ethyl)-piperazin-1-yl]-pyrimidin-4-ylamino}-thiazole-5-carboxylic acid [2-methyl-5-(3-trifluoromethyl-benzoylamino)-phenyl]-amide; 2-[2-Methyl-6-(4-methyl-piperazin-1-yl)-pyrimidin-4-ylamino]-thiazole-5-carboxylic acid [2-methyl-5-(3-trifluoromethyl-benzoylamino)-phenyl]-amide; and 2-{4-[4-(2-Hydroxy-ethyl)-piperazin-1-yl]-pyridin-2-ylamino}-thiazole-5-carboxylic acid [2-methyl-5-(3-trifluoromethyl-benzoylamino)-phenyl]-amide.

6. A pharmaceutical composition comprising a therapeutically effective amount of a compound of claim 1 in combination with a pharmaceutically acceptable excipient.

7. A method for treating a disease in an animal in which inhibition of kinase activity can prevent, inhibit or ameliorate the pathology and/or symptomology of the disease, which method comprises administering to the animal a therapeutically effective amount of a compound of claim 1.

8. The method of claim 8 in which the kinase is selected from the group consisting of Abl, Bcr-Abl, FGFR3, PDGFRβ and b-Raf.

9. The use of a compound of claim 1 in the manufacture of a medicament for treating a disease in an animal in which the kinase activity of Abl, Bcr-Abl, FGFR3, PDGFRβ and b-Raf contributes to the pathology and/or symptomology of the disease.
